(19) United States
(12) Patent Application Publication
(10) Pub. No.: US 2023/0273207 A1
(43) Pub. Date: Aug. 31, 2023

(54) ANTIGENS, ASSAYS, AND METHODS ASSOCIATED WITH SARS-COV-2

(71) Applicant: KREUTZ, Fernando Thome, Porto Alegre (BR)

(72) Inventor: Fernando Thome Kreutz, Porto Alegre (BR)

(21) Appl. No.: 18/005,429

(22) PCT Filed: Jul. 13, 2021
(86) PCT No.: PCT/IB2021/056314

§ 371 Date: Jan. 13, 2023

Related U.S. Application Data
(60) Provisional application No. 63/051,144, filed on Jul. 13, 2020.
(60) Provisional application No. 63/142,035, filed on Jan. 27, 2021.

(51) Int. Cl. G01N 33/569

(52) U.S. Cl. CPC G01N 33/56983; G01N 2333/165; G01N 2469/20

(57) ABSTRACT

Described herein is an assay for determining immune status in a subject. The assay comprises a probe for measuring disease-specific antibodies in a sample from the subject to determine a unit score. The unit score correlates to the immune status of the subject. Methods for determining the immune status of the subject are also described.

### FIELD

The present invention relates to antigens. More specifically, the present invention, is, in aspects, concerned with antigens for detecting and/or generating SARS-CoV-2 antibodies, assessment of immune status in relation to disease causing agents, and related methods, assays, and vaccines.

### BACKGROUND

The immune system is the body's first line defender against invasive pathogens. Defense against pathogens is mediated by the early reactions of the innate immune system and the later responses of the adaptive immune system. One arm of the adaptive immune response is humoral immunity, which is mediated by B cells that function by, for example, secreting antibodies. Secretion of these antibodies into the blood of a subject, can be detected using understood immunological methods, such as enzyme-linked immunosorbent assays.

While immunological components, like antibodies, can be detected, a reliable objective test to determine the immune status of a subject in relation to immunity to a pathogen remains be developed. Existence of a disease or infection is typically determined by a physician based on the subject's presented symptoms. Given the shared symptomology induced by different pathogenic agents, not only is the physician's diagnosis subjective, but without actually running biochemical tests, the causative agent and/or level of immunity (e.g., immune status) cannot be determined. In addition, rapid and reliable determination of a subject's immune status in relation to appropriateness for different treatments regimes (e.g. vaccines) is also unavailable; such testing could be useful in assessing whether or not the subject is a suitable candidate to receive a particular treatment.

Severe acute respiratory syndrome coronavirus 2 (SARS-CoV-2) is a strain of coronavirus that causes coronavirus disease 2019 (COVID-19), the respiratory illness responsible for the COVID-19 pandemic. Satarker and Nampoothiri (Archives of Medical Research (2020);

https://doi.org/10.1016/j.arcmed.2020.05.012) is a review article that describes the SARS-CoV-2 structural proteins.

Ou et al. (Nature Communications (2020) 11:1620; https://doi.org/10.1038/s41467-020-15562-9) describe using SARS-CoV-2 S protein pseudovirus system, to confirm that human angiotensin converting enzyme 2 (hACE2) is the receptor for SARS-CoV-2, and found that SARS-CoV-2 enters 293/hACE2 cells mainly through endocytosis, that PIKfyve, TPC2, and cathepsin L are critical for entry, and that SARS-CoV-2 S protein is less stable than SARS-CoV S. This reference also discloses that polyclonal anti-SARS S1 antibodies T62 inhibit entry of SARS-CoV S but not SARS-CoV-2 S pseudovirions and states that further studies using recovered SARS and COVID-19 patients' sera show limited cross-neutralization, suggesting that recovery from one infection might not protect against the other.

Ravichandran et al. (Sci. Transl. Med. (2020) 12:eabc3539) performed a qualitative study by immunizing rabbits with various SARS-CoV-2 spike protein antigens: S ectodomain (S1+S2; amino acids 16 to 1213), which lacks the cytoplasmic and transmembrane domains (CT-TM), the S1 domain (amino acids 16 to 685), the receptor binding domain (RBD) (amino acids 319 to 541), and the S2 domain (amino acids 686 to 1213, lacking the RBD, as control). Resulting antibody quality and function were analyzed by enzyme-linked immunosorbent assay (ELISA), RBD competition assay, surface plasmon resonance (SPR) against different spike proteins in native conformation, and neutralization assays. All three antigens (S1+S2 ectodomain, S1 domain, and RBD), but not S2, generated strong neutralizing antibodies against SARS-CoV-2. Vaccination-induced antibody repertoire was analyzed by SARS-CoV-2 spike genome fragment phage display libraries (SARS-CoV-2 GFPDL), which identified immunodominant epitopes in the S1, S1-RBD, and S2 domains. Furthermore, these analyses demonstrated that the RBD immunogen elicited a higher antibody titer with five-fold higher affinity antibodies to native spike antigens compared with other spike antigens, and antibody affinity correlated strongly with neutralization titers.

Long et al. (Nature Medicine (2020) https://doi.org/10.1038/s41591-020-0965-6) studied 37 asymptomatic individuals in the Wanzhou District who were diagnosed with RT-PCR-confirmed SARS-CoV-2 infections but without any relevant clinical symptoms in the preceding 14 d and during hospitalization. Asymptomatic individuals were admitted to the government-designated Wanzhou People's Hospital for centralized isolation in accordance with policy 1. The median duration of viral shedding in the asymptomatic group was 19 d (interquartile range (IQR), 15-26 d). The asymptomatic group had a significantly longer duration of viral shedding than the symptomatic group (log-rank P=0.028). The virus-specific IgG levels in the asymptomatic group (median S/CO, 3.4; IQR, 1.6-10.7) were significantly lower (P=0.005) relative to the symptomatic group (median S/CO, 20.5; IQR, 5.8-38.2) in the acute phase. Of asymptomatic individuals, 93.3% (28/30) and 81.1% (30/37) had reduction in IgG and neutralizing antibody levels, respectively, during the early convalescent phase, as compared to 96.8% (30/31) and 62.2% (23/37) of symptomatic patients. Forty percent of asymptomatic individuals became seronegative and 12.9% of the symptomatic group became negative for IgG in the early convalescent phase. In addition, asymptomatic individuals exhibited lower levels of 18 pro- and anti-inflammatory cytokines. These data suggest that asymptomatic individuals had a weaker immune response to SARS-CoV-2 infection. The reduction in IgG and neutralizing antibody levels in the early convalescent phase might have implications for immunity strategy and serological surveys.

Wrapp et al. (Science (2020), 367:1260-1263) determined a 3.5-angstrom-resolution cryo-electron microscopy structure of the 2019-nCoV S trimer in the prefusion conformation. The predominant state of the trimer has one of the three receptor-binding domains (RBDs) rotated up in a receptor-accessible conformation. This reference also provides biophysical and structural evidence that the 2019-nCoV S protein binds angiotensin-converting enzyme 2 (ACE2) with higher affinity than does severe acute respiratory syndrome (SARS)-CoV S. Additionally, several published SARS-CoV RBD-specific monoclonal antibodies were tested and it was found that they do not have appreciable binding to 2019-nCoV S, suggesting that antibody cross-reactivity may be limited between the two RBDs.

Walls et al. (Cell (2020), 180:281-292) show that SARS-CoV-2 S uses ACE2 to enter cells and that the receptor-binding domains of SARS-CoV-2 S and SARS-CoV S bind with similar affinities to human ACE2, correlating with the efficient spread of SARS-CoV-2 among humans. This reference found that the SARS-CoV-2 S glycoprotein harbors a furin cleavage site at the boundary between the S1/S2 subunits, which is processed during biogenesis and sets this virus apart from SARS-CoV and SARS-related CoVs. The cryo-EM structures of the SARS-CoV-2 S ectodomain trimer were determined, providing a blueprint for the design of vaccines and inhibitors of viral entry. Finally, it was demonstrated that SARS-CoV S murine polyclonal antibodies potently inhibited SARS-CoV-2 S mediated entry into cells, indicating that cross-neutralizing antibodies targeting conserved S epitopes can be elicited upon vaccination.

SARS-CoV-2 is a quickly growing concern worldwide and testing and vaccine development are both critically needed. Furthermore, a need exists for the development of an assay and related methods for predicting immune status of a subject.

### SUMMARY

In accordance with an aspect, there is provided an assay for determining immune status in a subject, the assay comprising: a probe for measuring disease-specific antibodies in a sample from the subject to determine a unit score, wherein the unit score correlates to the immune status of the subject.

In aspects, the unit score comprises an U-IBBR value.

In aspects, the U-IBBR value is predictive of the immune status of the subject.

In aspects, the U-IBBR value stratifies the subject into an immune status group.

In aspects, the immune status group comprises a low immunity group and a high immunity group.

In aspects, the immune status group comprises two or more of a low immunity group, a low-moderate immunity group, a moderate-high immunity group, and a high immunity group.

In aspects, the immune status group comprises a low immunity group, a low-moderate immunity group, a moderate-high immunity group, and a high immunity group.

In aspects, the U-IBBR value ranges from 0 to about 300.

In aspects, the U-IBBR value ranges from 0 to about 200.

In aspects, the U-IBBR value ranges from 0 to about 90 in the low immunity group and the U-IBBR value ranges from about 110 to about 300 in the high immunity group.

In aspects, the U-IBBR value ranges from 20 to about 70 in the low immunity group and the U-IBBR value ranges from about 140 to about 300 in the high immunity group.

In aspects, the U-IBBR value ranges from about 30 to about 60 in the low immunity group and the U-IBBR value ranges from about 140 to about 300 in the high immunity group.

In aspects, the U-IBBR value ranges from 0 to about 50 in the low immunity group, the U-IBBR value ranges from about 51 to about 100 in the low-moderate immunity group, the U-IBBR value ranges from about 101 to about 140 in the moderate-high immunity group and the U-IBBR value ranges from about 141 to about 300 in the high immunity group.

In aspects, the U-IBBR value ranges from 0 to about 50 in the low immunity group, the U-IBBR value ranges from about 40 to about 100 in the low-moderate immunity group, the U-IBBR value ranges from about 70 to about 150 in the moderate-high immunity group and the U-IBBR value ranges from about 130 to about 300 in the high immunity group.

In aspects, the U-IBBR value ranges from 0 to about 90 in the low immunity group and the U-IBBR value ranges from about 110 to about 200 in the high immunity group.

In aspects, the U-IBBR value ranges from 20 to about 70 in the low immunity group and the U-IBBR value ranges from about 140 to about 200 in the high immunity group.

In aspects, the U-IBBR value ranges from about 30 to about 60 in the low immunity group and the U-IBBR value ranges from about 140 to about 200 in the high immunity group.

In aspects, the U-IBBR value ranges from 0 to about 50 in the low immunity group, the U-IBBR value ranges from about 51 to about 100 in the low-moderate immunity group, the U-IBBR value ranges from about 101 to about 140 in the moderate-high immunity group and the U-IBBR value ranges from about 141 to about 200 in the high immunity group.

In aspects, the U-IBBR value ranges from 0 to about 50 in the low immunity group, the U-IBBR value ranges from about 40 to about 100 in the low-moderate immunity group, the U-IBBR value ranges from about 70 to about 150 in the moderate-high immunity group and the U-IBBR value ranges from about 130 to about 200 in the high immunity group.

In aspects, the probe comprises an antigen.

In aspects, the antigen comprises a SARS-CoV-2 S1 or S2 protein or a variant or combination thereof.

In aspects, the antigen comprises or consists of the polypeptide sequence of SEQ ID NO:1 or a variant thereof.

In aspects, the immune status is related to a disease of the subject.

In aspects, the disease is an infectious disease or a non-infectious disease.

In aspects, the infectious disease is selected from hepatitis, strep throat, urinary tract infections, tuberculosis, malaria, dengue fever, meningococcal disease, chloera, rabies, ebola, influenza, coronavirus disease-2019 (COVID-19), herpes, respiratory infection, Severe Acute Respiratory Syndrome (SARS), Middle East Respiratory Syndrome (MERS), mumps, measles, rubella, polio, small pox, human immunodeficiency virus (HIV), acquired immune deficiency syndrome (AIDS), pneumonia, Lyme disease, anthrax, tetanus, cholera, plague, diptheria, chlamydia or malaria.

In aspects, the infectious disease is coronavirus disease-2019 (COVID-19).

In aspects, the sample is selected from blood, blood plasma, blood serum, hemolysate, spinal fluid, urine, lymph, synovial fluid, saliva, sperm, amniotic fluid, lacrimal fluid, cyst fluid, sweat gland secretion, or bile.

In aspects, the sample is blood.

In accordance with another aspect, there is provided a method for determining immune status in a subject, the method comprising: measuring disease-specific antibodies in a sample; and determining a unit score based on the disease-specific antibodies measured; wherein the unit score correlates to the immune status of the subject.

In aspects, the unit score comprises an U-IBBR value.

In aspects, the U-IBBR value is predictive of the immune status of the subject.

In aspects, the U-IBBR value stratifies the subject into an immune status group.

In aspects, the immune status group comprises a low immunity group and a high immunity group.

In aspects, the immune status group comprises two or more of a low immunity group, a low-moderate immunity group, a moderate-high immunity group, and a high immunity group.

In aspects, the immune status group comprises a low immunity group, a low-moderate immunity group, a moderate-high immunity group, and a high immunity group.

In aspects, the U-IBBR value ranges from 0 to about 300.

In aspects, the U-IBBR value ranges from 0 to about 200.

In aspects, the U-IBBR value ranges from 0 to about 90 in the low immunity group and the U-IBBR value ranges from about 110 to about 300 in the high immunity group.

In aspects, the U-IBBR value ranges from 20 to about 70 in the low immunity group and the U-IBBR value ranges from about 140 to about 300 in the high immunity group.

In aspects, the U-IBBR value ranges from about 30 to about 60 in the low immunity group and the U-IBBR value ranges from about 140 to about 300 in the high immunity group.

In aspects, the U-IBBR value ranges from 0 to about 50 in the low immunity group, the U-IBBR value ranges from about 51 to about 100 in the low-moderate immunity group, the U-IBBR value ranges from about 101 to about 140 in the moderate-high immunity group and the U-IBBR value ranges from about 141 to about 300 in the high immunity group.

In aspects, the U-IBBR value ranges from 0 to about 50 in the low immunity group, the U-IBBR value ranges from about 40 to about 100 in the low-moderate immunity group, the U-IBBR value ranges from about 70 to about 150 in the moderate-high immunity group and the U-IBBR value ranges from about 130 to about 300 in the high immunity group.

In aspects, the U-IBBR value ranges from 0 to about 90 in the low immunity group and the U-IBBR value ranges from about 110 to about 200 in the high immunity group.

In aspects, the U-IBBR value ranges from 20 to about 70 in the low immunity group and the U-IBBR value ranges from about 140 to about 200 in the high immunity group.

In aspects, the U-IBBR value ranges from about 30 to about 60 in the low immunity group and the U-IBBR value ranges from about 140 to about 200 in the high immunity group.

In aspects, the U-IBBR value ranges from 0 to about 50 in the low immunity group, the U-IBBR value ranges from about 51 to about 100 in the low-moderate immunity group, the U-IBBR value ranges from about 101 to about 140 in the moderate-high immunity group and the U-IBBR value ranges from about 141 to about 200 in the high immunity group.

In aspects, the U-IBBR value ranges from 0 to about 50 in the low immunity group, the U-IBBR value ranges from about 40 to about 100 in the low-moderate immunity group, the U-IBBR value ranges from about 70 to about 150 in the moderate-high immunity group and the U-IBBR value ranges from about 130 to about 200 in the high immunity group.

In aspects, the probe comprises an antigen.

In aspects, the antigen comprises a SARS-CoV-2 S1 or S2 protein or a variant or combination thereof.

In aspects, the antigen comprises or consists of the polypeptide sequence of SEQ ID NO:1 or a variant thereof.

In aspects, the immune status is related to a disease of the subject.

In aspects, the disease is an infectious disease or a non-infectious disease.

In aspects, the infectious disease is selected from hepatitis, strep throat, urinary tract infections, tuberculosis, malaria, dengue fever, meningococcal disease, chloera, rabies, ebola, influenza, coronavirus disease-2019 (COVID-19), herpes, respiratory infection, Severe Acute Respiratory Syndrome (SARS), Middle East Respiratory Syndrome (MERS), mumps, measles, rubella, polio, small pox, human immunodeficiency virus (HIV), acquired immune deficiency syndrome (AIDS), pneumonia, Lyme disease, anthrax, tetanus, cholera, plague, diptheria, chlamydia or malaria.

In aspects, the infectious disease is coronavirus disease-2019 (COVID-19).

In aspects, the sample is selected from blood, blood plasma, blood serum, hemolysate, spinal fluid, urine, lymph, synovial fluid, saliva, sperm, amniotic fluid, lacrimal fluid, cyst fluid, sweat gland secretion, or bile.

In aspects, the sample is blood.

In aspects, the method described herein is useful for predicting the likelihood of being infected with a pathogen.

In aspects, the method described herein is useful for determining vaccination status for the disease.

In aspects, the method described herein is useful for determining whether the subject should be administered an RNA vaccine against the disease.

In aspects, the method described herein further comprises vaccinating the subject if the immune status has a predetermined unit score value.

In aspects, the predetermined unit score value ranges from 0 to about 140.

In accordance with another aspect, there is provided a method for predicting the likelihood of being infected with a pathogen, the method comprising: measuring pathogen-specific antibodies in a sample; and determining a unit score based on the pathogen-specific antibodies measured; wherein the unit score predicts the likelihood of being infected with the pathogen.

In accordance with yet another aspect, there is provided a method for determining vaccination status for a disease, the method comprising: measuring disease-specific antibodies in a sample from a subject; and determining a unit score based on the disease-specific antibodies measured; wherein the unit score correlates to vaccination status.

In accordance with yet another aspect, there is provided a method for determining whether a subject should be administered an RNA vaccine against a disease, the method comprising: measuring disease-specific antibodies in a sample from a subject; and determining a unit score based on the disease-specific antibodies measured; wherein the unit score predicts whether the subject should receive the RNA vaccine.

In accordance with still another aspect, there is provided a method for vaccinating a subject against a disease, the method comprising: measuring disease-specific antibodies in a sample from the subject; determining a unit score based on the disease-specific antibodies measured; wherein the unit score correlates to an immune status of the subject; and vaccinating the subject if the immune status has a predetermined unit score value.

In an aspect, there is provided a method for measuring vaccine efficacy, the method comprising: measuring disease-specific antibodies in a sample from a vaccinated subject; and determining a unit score based on the disease-specific antibodies measured; wherein the unit score predicts the efficacy of the vaccine.

In aspects, the methods described herein comprise a step of obtaining the sample from the subject.

In accordance with yet another aspect, there is provided an antigen for detecting and/or generating SARS-CoV-2 antibodies, wherein the antigen comprises the SARS-CoV-2 S1 and S2 proteins or variants thereof, wherein the S1 and S2 proteins or variants adopt a substantially native conformation.

In accordance with yet another aspect, there is provided an antigen for detecting and/or generating SARS-CoV-2 antibodies, wherein the antigen comprises a spike protein conformational epitope or variant thereof, wherein the epitope is not present in S1 or S2 alone.

In accordance with still yet another aspect, there is provided an antigen comprising or consisting of the polypeptide sequence of SEQ ID NO:1:

| | |
|---|---|
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfcndpf lgvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqdvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkfngltv lpplltdemi aqytsallag titsgwtfga gaalqipfam |
| 901 | qmayrfngig vtqnvlyenq klianqfnsa igkiqdslss tasalgklqd vvnqnaqaln |
| 961 | tlvkqlssnf gaissvlndi lsrldkveae vqidrlitgr lqslqtyvtq qliraaeina |
| 1021 | sanlaatkms ecvlgqskrv dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyeq |

or a variant thereof.

In an aspect, the antigen further comprises one or more stabilizing residues, such as stabilizing proline residues, for example at the C-terminus.

In an aspect, the variant comprises at least about 50%, 60%, 70%, 80%, 90%, 95%, or 99% sequence identity to the native S1 and S2 proteins, the native conformational epitope, or SEQ ID NO:1.

In an aspect, the antigen further comprises the SARS-CoV-2 trunk protein.

In an aspect, the antigen is N-glycosylated.

In an aspect, the antigen is N-glycosylated at 50%, 60%, 70%, 80%, 90%, 95%, or 99%, or more, or 100% of the native N-glycosylation sites.

In an aspect, the antigen is trimeric.

In accordance with still yet another aspect, there is provided a diagnostic assay comprising the antigen described herein.

In an aspect, the assay is an ELISA, a lateral flow assay, or a chemiluminescence immunoassay.

In an aspect, the assay detects IgG, IgM, and/or IgA.

In an aspect, the assay detects antibodies in a sample selected from blood, blood plasma, blood serum, hemolysate, spinal fluid, urine, lymph, synovial fluid, saliva, sperm, amniotic fluid, lacrimal fluid, cyst fluid, sweat gland secretion, and bile.

In accordance with still yet another aspect, there is provided a vaccine against SARS-CoV-2 comprising the antigen described herein.

In an aspect, the antigen further comprises an adjuvant.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 shows coating optimization experiments. (A) dose response for S1 antigen concentration used for positive sample 1; (B) dose response for S1 antigen concentration used for positive sample 2; (C) dose response for S1 antigen concentration using a pool of negative samples.

FIG. 2 shows dose response curves for S total and S1 antigens on two different positive samples and a pool of negative samples

FIG. 3 shows the coating of S total protein at different concentrations. (A) results using various concentrations of S total protein as compared to S1 at 1 ug/ml and a BSA control; (B) results of S total at various concentrations using a pool of positive samples; (C) results of S total at various concentrations using a pool of negative samples.

FIG. 4 shows the results of testing to determine appropriate amounts of secondary antibody to use in subsequent testing.

FIG. 5 shows cutoff determination assays using S1 and S total. (A) results with S total; (B) results with S1.

FIG. 6 shows the comparison between positive and negative samples using S total as the antigen.

FIG. 7 shows patient sample stability results. (A) correlation between stability at 4° C. and 37° C.; (B) bar graphs showing stability.

FIG. 8 shows the coefficient of variation for S total antigen.

FIG. 9 shows that assays done using separate plates and operators had no significant variation.

FIG. 10 shows sample titration results. (A) first set of patient samples; (B) second set of patient samples.

FIG. 11 shows assay results for Patient Cluster 1.

FIG. 12 shows assay results for Patient Cluster 2.

FIG. 13 shows assay results for Patient Cluster 3.

FIG. 14 shows assay results over time for a group of co-workers at a medical clinic.

FIG. 15 shows a comparison of results between S1 and S total across a number of different patients. (A) results for Patient A; (B) results for Patient B; (C) results for Patient C.

FIG. 16 shows a schematic relating to FIG. 15.

FIG. 17 shows a comparison of results between S1 and S total across a number of different patients. (A) results for Patient D; (B) results for Patient E; (C) results for Patient F.

FIG. 18 shows a schematic relating to FIG. 17.

FIG. 19 shows a comparison of results between S1 and S total across a number of different patients. (A) results for Patient G; (B) results for Patient H; (C) results for Patient K.

FIG. 20 shows a schematic relating to FIG. 19.

FIG. 21 shows the correlation between the O.D. for S total and S1 for a number of patients.

FIG. 22 shows a plot of the U-IBBR unit level results from the serum of 1224 pre-COVID-19 subjects.

FIG. 23 shows a distribution plot of healthy pre-COVID-19 patients verses U-IBBR unit level from the data of FIG. 22.

FIG. 24 shows distribution plots of healthy pre-COVID-19 patients verses U-IBBR unit level from the data of FIG. 23 wherein different sub-populations of patients are identified based on their U-IBBR unit level (white bar represents undetectable humoral response, grey bar represents a first level of cross reactive humoral response and black bar represents a second level of cross reactive humoral response).

FIG. 25 shows a plot of the results of healthy pre-COVID-19 patients verses U-IBBR unit level to represent the sub-populations of FIG. 24 including the humoral response of a fourth group of patients that tested positive for COVID-19.

FIG. 26 shows a graph of U-IBBR unit level verses humoral response detected illustrating the probability of belonging to one of the four groups identified in FIG. 25.

FIG. 27 shows a plot demonstrating U-IBBR unit level pre-COVID-19 exposure (left side) verses U-IBBR unit level post exposure/recovery from COVID-19 (right side) with time on the x axis and U-IBBR value on the y axis.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications may be referred to herein to assist in understanding the aspects described. Each of these references is incorporated herein by reference in its entirety.

When introducing elements disclosed herein, the articles “a”, “an”, “the”, and “said” are intended to mean that there may be one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any embodiments described as “comprising” certain components may also “consist of” or “consist essentially of,” these components, wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects described herein. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation, such as any specific compounds or method steps, whether implicitly or explicitly defined herein.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Although methods and material similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

“Immune status” herein refers to the ability of the body to demonstrate an immune response (e.g. a humoral response or a cellular response) or to defend itself against disease caused by a disease causing agent/infectious agent (e.g., a microorganism) or a foreign substance (e.g. a microorganism or any part thereof).

As used herein, the terms “disease” and “disorder” are used interchangeably to refer to a condition, in particular, a pathological condition.

The immune status of infectious diseases that can assessed using the assay and methods described herein are caused by infectious agents including, but not limited to, bacteria, fungi, protozoa, and viruses. Viral diseases include, but are not limited to, those caused by rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-I), human immunodeficiency virus type II (HIV-II) and agents of viral diseases such as viral miningitis, encephalitis, dengue, small pox, hepatitis type A virus, hepatitis type B virus, hepatitis type C virus, influenza (e.g., influenza A or influenza B) virus, herpes simplex type I (HSV-I) virus, and herpes simplex type II (HSV-II) virus and SARS-CoV-2.

Bacterial diseases include, but are not limited to, those caused by Escherichia coli, Klebsiella pneumoniae, Staphylococcus aureus, Enterococcus faecials, Proteus vulgaris, Staphylococcus viridans, and Pseudomonas aeruginosa, mycobacteria rickettsia, mycoplasma, neisseria, S. pneumonia, Borrelia burgdorferi (Lyme disease), Bacillus antracis (anthrax), tetanus, streptococcus, staphylococcus, mycobacterium, pertissus, Vibrio cholerae (cholera), Yersinia pestis (plague), Corynebacterium diphtheriae (diptheria), chlamydia trachomatis, S. aureus and legionella.

Protozoan diseases include, but are not limited to, those caused by, leishmania, kokzidioa, or trypanosoma schistosoma. Parasitic diseases caused by parasites include, but are not limited to, malaria.

The immune status of non-infectious diseases that can assessed using the assay and methods described herein are caused by non-infectious agents, such as autoantigens, food antigens and the like, and the non-infectious diseases include, but are not limited to, cancer, allergic disease, cardiovascular disease, cardiac disease, a disease of the central nervous system, diabetes, autoimmune disorder, and a disorder associated with inflammation.

The terms “increased” or “increased expression” and “decreased” or “decreased expression”, with respect to the expression pattern of an antibody are used herein as meaning that the level of expression is increased or decreased relative to a constant basal level of expression of a household, or housekeeping, protein, whose expression level does not significantly vary under different conditions. A nonlimiting example of such a household, or housekeeping, protein is GAPDH. Other suitable household, or housekeeping, proteins are well-established in the art. In other aspects, these terms refer to an increase or decrease in the level of expression as compared to that observed in a control population, such as a subject or pool of subjects who are SARS-CoV-2 negative, or a known negative sample. In other aspects, these terms refer to an increase or decrease in relative concentrations in relation to the mean values of the sample in question.

“Variants” of the sequences described herein are biologically active sequences that have a peptide sequence that differs from the sequence of a native or wild-type sequence, by virtue of an insertion, deletion, modification and/or substitution of one or more amino acids within the native sequence. Such variants generally have less than 100% sequence identity with a native sequence. Ordinarily, however, a biologically active variant will have an amino acid sequence with at least about 70% sequence identity with the sequence of a corresponding naturally occurring sequence, typically at least about 75%, more typically at least about 80%, even more typically at least about 85%, even more typically at least about 90%, and even more typically of at least about 95%, 96%, 97%, 98%, or 99% sequence identity. The variants fragments of any length that retain a biological activity of the corresponding native sequence. Variants also include sequences wherein one or more amino acids are added at either end of, or within, a native sequence. Variants also include sequences where a number of amino acids are deleted and optionally substituted by one or more different amino acids.

“Percent sequence identity” is defined herein as the percentage of amino acid residues in the candidate sequence that are identical with the residues in the sequence of interest after aligning the sequences and introducing gaps, if necessary, to achieve the maximum percent sequence identity, and not considering any conservative substitutions as part of the sequence identity. None of 5′, 3′, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological activity of a native or naturally-occurring antigen, wherein “biological” activity refers to a biological function (either inhibitory or stimulatory) caused by a native or naturally-occurring antigen.

Thus, “biologically active” or “biological activity” when used in conjunction with the antigens described herein refers to an antigen or amino acid sequence that exhibits or shares an effector function of the native antigen or sequence.

“Biologically active” or “biological activity” when used in conjunction with variant sequences means that the variant sequences exhibit or share an effector function of the parent sequence. The biological activity of the variant sequence may be increased, decreased, or at the same level as compared with the parent sequence.

“Isolated” refers to a molecule that has been purified from its source or has been prepared by recombinant or synthetic methods and purified. Purified antigens are substantially free of other amino acids.

“Substantially free” herein means less than about 5%, typically less than about 2%, more typically less than about 1%, even more typically less than about 0.5%, most typically less than about 0.1% contamination with other source amino acids. An “essentially pure” antigen composition means a composition comprising at least about 90% by weight of the antigen, based on total weight of the composition, typically at least about 95% by weight, more typically at least about 90% by weight, even more typically at least about 95% by weight, and even more typically at least about 99% by weight of the antigen, based on total weight of the composition.

As used herein, “treatment” or “therapy” is an approach for obtaining beneficial or desired clinical results. For the purposes described herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” and “therapy” can also mean prolonging survival as compared to expected survival if not receiving treatment or therapy. Thus, “treatment” or “therapy” is an intervention performed with the intention of altering the pathology of a disorder. Specifically, the treatment or therapy may directly prevent, slow down or otherwise decrease the pathology of a disease or disorder such as an infection, or may render the infection more susceptible to treatment or therapy by other therapeutic agents.

The terms “therapeutically effective amount”, “effective amount” or “sufficient amount” mean a quantity sufficient, when administered to a subject, including a mammal, for example a human, to achieve a desired result, for example an amount effective to treat an infection. Effective amounts as described herein may vary according to factors such as the disease state, age, sex, and weight of the subject. Dosage or treatment regimes may be adjusted to provide the optimum therapeutic response, as is understood by a skilled person.

Moreover, a treatment regime of a subject with a therapeutically effective amount may consist of a single administration, or alternatively comprise a series of applications. The length of the treatment period depends on a variety of factors, such as the severity and/or site of the disease, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The antigens described herein may, in aspects, be administered before, during or after treatment with conventional therapies for the disease or disorder in question, such as an infection.

The term “subject” as used herein refers to any member of the animal kingdom, including birds, fish, invertebrates, amphibians, mammals, and reptiles. Typically, the subject is a human or non-human vertebrate. Non-human vertebrates include livestock animals, companion animals, and laboratory animals. Non-human subjects also specifically include non-human primates as well as rodents. Non-human subjects also specifically include, without limitation, poultry, chickens, horses, cows, pigs, goats, dogs, cats, guinea pigs, hamsters, mink, rabbits, crustaceans, and molluscs. Typically the subject is poultry or a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

“Carriers” as used herein include pharmaceutically acceptable carriers, excipients, or stabilizers that are nontoxic to the cell or subject being exposed thereto at the dosages and concentrations employed. Often the pharmaceutically acceptable carrier is an aqueous pH buffered solution. Examples of pharmacologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, and dextrins; chelating agents such as EDTA; sugar alcohols such as mannitol and sorbitol; salt-forming counterions such as sodium; and/or nonionic surfactants such as TWEEN™, polyethylene glycol (PEG), and PLURONICS™.

### Antigens, Assays, and Methods for Detecting and/or Generating SARS-CoV2 Antibodies

Described herein is an antigen for detecting and/or generating SARS-CoV-2 antibodies. The antigen typically comprises SARS-CoV-2 S1 and S2 proteins or variants thereof, wherein the S1 and S2 proteins or variants adopt a substantially native conformation. In this way, the conformation of the epitope that is used to detect and/or generate antibodies is biologically relevant and is more likely to detect and/or or generate biologically relevant antibodies.

In additional or alternative aspects, the antigen comprises a spike protein conformational epitope or variant thereof, wherein the epitope is not present in S1 or S2 alone. In this aspect, the epitope is an epitope of the spike protein that is not a cryptic epitope. It is an epitope that is exposed in the formed spike protein, either alone or in trimeric form.

In additional or alternative aspects, the antigen comprises, consists essentially of, or consists of the polypeptide sequence of SEQ ID NO:1:

| | |
|---|---|
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfcndpf lgvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqdvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkfngltv lpplltdemi aqytsallag titsgwtfga gaalqipfam |
| 901 | qmayrfngig vtqnvlyenq klianqfnsa igkiqdslss tasalgklqd vvnqnaqaln |
| 961 | tlvkqlssnf gaissvlndi lsrldkveae vqidrlitgr lqslqtyvtq qliraaeira |
| 1021 | sanlaatkms ecvlgqskrv dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyeq |

or a variant thereof. It will be understood that variants include fragments as described herein and that the variants typically retain the native conformation of the peptide in question.

The sequences may further comprise additional features, such as additional sequences as fusion proteins, stabilizing residues, such as proline residues, targeting sequences, or other such variations known to the skilled person to impact efficacy and/or potency for detecting and/or generating antibodies. For example, typically, a pair of proline residues are added at the C-terminus to improve stability. In other typical aspects, the trunk protein of SARS-CoV-2 is included in the sequence.

Typically, the variant comprises at least about 50%, at least about 60%, at least about 70%, at least about 80%, at least about 90%, at least about 95%, or at least about 99% sequence identity to the native S1 and S2 proteins, the native conformational epitope, or SEQ ID NO:1.

It will be understood that these proteins are typically glycosylated in vivo and the recombinant versions described herein may or may not be similarly glycosylated. Typically, however, the antigen is N-glycosylated. The antigen may be partially or fully N-glycosylated and it may be synthetically N-glycosylated or naturally N-glycosylated for example through production in mammalian cells. The antigen is, for example, N-glycosylated at about 50%, about 60%, about 70%, about 80%, about 90%, about 95%, or about 99%, or more, or 100% of the native N-glycosylation sites.

It will be understood that the S protein of SARS-CoV-2 is trimeric. The antigen thus is also typically trimeric in the diagnostic assays and/or vaccinations described herein. The antigen, however, can be in monomeric form or other oligomeric forms, provided the conformational epitope(s) remain in their substantially native conformation.

Also described herein is a diagnostic assay comprising the antigen described herein. The assay may be of any form that detects antibodies. Typically, the assay is an ELISA, a lateral flow assay, or a chemiluminescence immunoassay. The assay can detect any type of antibody, depending on the sample in question, but typically the assay detects IgG, IgM, and/or IgA. The sample tested in the assay may be any fluid, such as a bodily fluid, such as blood, blood plasma, blood serum, hemolysate, spinal fluid, urine, lymph, synovial fluid, saliva, sperm, amniotic fluid, lacrimal fluid, cyst fluid, sweat gland secretion, and bile. Typically, the sample is a blood sample.

Also described herein is a vaccine against SARS-CoV-2 comprising the antigen described herein. Typically, the vaccine comprises an adjuvant as will be understood.

### Assays and Methods for Determining Immune Status

Assays and methods of determining immune status of a subject are described herein. In typical aspects, an assay for determining immune status in a subject comprises a probe, such as an antigen, for measuring disease-specific antibodies in a sample from the subject to determine a unit score (e.g. U-IBBR value, also see Example 2). In other aspects, a method for determining immune status in the subject comprises measuring disease-specific antibodies in the sample and determining the unit score based on the disease-specific antibodies measured. Typically, the unit score is correlated to the immune status of the subject. In this way, the unit score is useful for the prediction of the immune status (e.g., level of protective immunity) of the subject.

In typical aspects, the U-IBBR value is capable of stratifying the subject into an immune status group. The immune status group stratification, in typical aspects, relates to a spectrum of protection against an invading pathogen, for example, ranging from no immunity to complete immunity. In typical aspects, the immune status group comprises an immunity group which is denoted by the subject's immunity status to the antigen (e.g., the level of immune protection of the subject). In this way, the subject can be placed into the immunity group which relates to their level of protection from said pathogen, based on, for example, the measure of the disease-specific antibodies against said pathogen. Thus, the assays and methods described herein can provide for the determination of the subject's level of exposure to said pathogen on an increasing gradient of the same.

In typical aspects, the immunity group can be a low immunity group, a low-moderate immunity group, a moderate-high immunity group and a high immunity group. In typical aspects, the immune status group comprises the low immunity group and the high immunity group. In further aspects, the immune status group comprises two or more of the low immunity group, the low-moderate immunity group, the moderate-high immunity group, and the high immunity group. In even further aspects, the immune status group comprises the low immunity group, the low-moderate immunity group, the moderate-high immunity group, and the high immunity group.

In this way, as a subject moves from the low immunity group to the high immunity group, or if one is comparing the results of a subject in the low immunity group and a subject in the high immunity group, the level of protection against the disease-causing pathogen increases from, for example, no and/or low immunity (i.e., “low immunity” group) to complete immunity in respect of that pathogen (i.e., “high immunity” group). The former group would have, for example, either no or undetectable humoral responses in respect of the pathogen of interest. In between the extremes of the stratified groups, subject's falling within the low-moderate immunity and the moderate-high immunity groups, have humoral responses which are, in most cases, related to cross-reactive antigens (e.g., results related to the subject's prior exposure to viral antigens other than the ones being tested).

Based on the scoring system described herein, methods related to predicting the likelihood of being infected with a pathogen are also described. This method comprises measuring pathogen-specific antibodies in the sample and determining the unit score based on the pathogen-specific antibodies measured. The unit score predicts the likelihood of being infected with the pathogen. Using the example provided above, the subject with a unit score falling within the low immunity group would not be considered likely to have seen the pathogen, whereas the subject having a unit score falling within the high immunity group is considered immune to the pathogen in question. Subject's falling within the other two groups have likely been exposed to similar albeit not the same pathogen and therefore produce a detectable humoral response.

Since these immunity groups are differentiated by the unit score value that is obtained from, for example, the measurement of the disease-specific antibodies in the sample of the subject, placing the subject in one of the low immunity group, the low-moderate immunity group, the moderate-high immunity group, and the high immunity group, may be a way to discern individuals who stand a better chance of warding off, for example, an infectious agent, especially when dealing with an impending pandemic. For example, a subject who has never been exposed to a particular virus, such as, SARS-CoV-2, responsible for coronavirus disease-2019 (COVID-19), would be considered to have no and/or low immunity to this virus and would, based on their unit score, fall into the “low immunity” group category. Alternatively, if another subject were exposed to the same virus and built an immune response against it such that they have recovered from the virus and/or have developed a memory-type response, this subject would be in the “high immunity” group category. It could be said that with the increased restrictions on travel, outdoor and/or socializing activities allowable in connection to COVID-19, the assays and methods described herein, may allow for increased awareness of one's exposure and/or immunity to the SARS-CoV-2 virus, such that stress levels surrounding re-exposure for the high immunity group subjects may be limited. In this way, the assays and methods described herein may play a role in providing a sort of “COVID passport”, wherein identified immune individuals (while still engaging in the required COVID-related practices, e.g., mask wearing and hand washing), would be able to put themselves in more dangerous situations (e.g., front line worker).

In typical aspects, the unit score (e.g. U-IBBR value) ranges from 0 to about 300 across the low immunity group, the low-moderate immunity group, the moderate-high immunity group and the high immunity group. For example, the U-IBBR value can be 0, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, and about 300. In more typical aspects, the U-IBBR value ranges from 0 to about 200. In aspects, there is an overlap in the unit score as it pertains to the immunity group. For example, the U-IBBR value of the low immunity group may range from 0 to about 50, the U-IBBR value of the low-moderate immunity group may range from about 40 to about 100 group, the U-IBBR value of the moderate-high immunity group may range from about 70 to about 150 and the U-IBBR value of the high immunity group may range from about 130 to about 200. In another example, the U-IBBR value of the low immunity group may range from 0 to about 50, the U-IBBR value of the low-moderate immunity group may range from about 40 to about 100 group, the U-IBBR value of the moderate-high immunity group may range from about 70 to about 150 and the U-IBBR value of the high immunity group may range from about 130 to about 300. In other aspects, there may be minimal overlap between the groups, such as for example, the U-IBBR value of the low immunity group may range from 0 to about 50, the U-IBBR value in the low-moderate group may range from about 51 to about 100, the U-IBBR value of the moderate-high immunity group may range from about 101 to about 140 and the U-IBBR value of the high immunity group may range from about 141 to about 200. In another example, the U-IBBR value of the low immunity group may range from 0 to about 50, the U-IBBR value in the low-moderate group may range from about 51 to about 100, the U-IBBR value of the moderate-high immunity group may range from about 101 to about 140 and the U-IBBR value of the high immunity group may range from about 141 to about 300.

There is typically no overlap in the unit score values between the low immunity group and the high immunity group. For example, using the range from 0 to about 300, the U-IBBR value for the low immunity group may range from 0 to about 90 whereas the U-IBBR value of the high immunity group may range from about 110 to about 300. In another example, the U-IBBR value for the low immunity group may range from 0 to about 90 whereas the U-IBBR value of the high immunity group may range from about 140 to about 300. In other examples, the U-IBBR value of the low immunity group may range from 20 to about 70 whereas the U-IBBR value of the high immunity group may range from about 110 to about 300. In other examples, the U-IBBR value of the low immunity group may range from 20 to about 70 whereas the U-IBBR value of the high immunity group may range from about 140 to about 300. In another example, the U-IBBR value of the low immunity group may range from about 30 to about 60 and the U-IBBR value of the high-immunity group may range from about 110 to about 300. In another example, the U-IBBR value of the low immunity group may range from about 30 to about 60 and the U-IBBR value of the high-immunity group may range from about 140 to about 300.

In a different example, using the range from 0 to about 200, the U-IBBR value for the low immunity group may range from 0 to about 90 whereas the U-IBBR value of the high immunity group may range from about 110 to about 200. In another example, the U-IBBR value for the low immunity group may range from 0 to about 90 whereas the U-IBBR value of the high immunity group may range from about 140 to about 200. In other examples, the U-IBBR value of the low immunity group may range from 20 to about 70 whereas the U-IBBR value of the high immunity group may range from about 110 to about 200. In other examples, the U-IBBR value of the low immunity group may range from 20 to about 70 whereas the U-IBBR value of the high immunity group may range from about 140 to about 200. In another example, the U-IBBR value of the low immunity group may range from about 30 to about 60 and the U-IBBR value of the high-immunity group may range from about 110 to about 200. In another example, the U-IBBR value of the low immunity group may range from about 30 to about 60 and the U-IBBR value of the high-immunity group may range from about 140 to about 200. Typically, the U-IBBR value for the low immunity group ranges from 0 to about 90, more typically between about 30 and about 60; and the U-IBBR value for the high immunity group ranges from about 110 to about 200, more typically between about 150 to about 200.

In particular reference to the overlapping ranges of the U-IBBR values described herein, the algorithm provides for a prediction of the probability that the calculated unit score is connected with a single immune status group (see Table 8 for the calculated probabilities). For example, referring to Table 8, an individual with a score of 30 is 100% likely/predicted to be in group 1 (undetectable humoral response and therefore has low or no immunity to the virus). A subject with a unit score of 80 is 97.60% likely to be in group 2 (humoral response cross reactive level I) and only 1.66% likely to be in group 3 (humoral response cross reactive group II). A subject with a unit score of 130 is 89.62% likely to be in group 3 (humoral response cross reactive level II) and only 10.38% likely to be group 4 (pos-COVID-19 humoral response). Finally, a subject with a unit score of 180 or better is 100% likely/predicted to be in group 4 (pos-COVID-19 humoral response). Based on this hypothetical scoring system, the subject with the unit score of 180 or better would be a potential candidate, for example, to be put on, and not suffer from exposure to, the front lines (e.g., medical professional treating infected and suffering patients).

The assays and methods using the same scoring and stratification system described herein, may also be useful in identifying subject's candidacy for particular treatments. In this way, methods are provided for determining vaccination status for a disease, the method comprising measuring disease-specific antibodies in the sample from the subject and determining the unit score based on the disease-specific antibodies measured. In this method, the unit score correlates to vaccination status. For example, if the subject's unit score falls within the low immunity group, this subject has not received the vaccine as an appreciable humoral response is not detectable by the assay described herein. However, if the subject's unit score falls within the high immunity group, the subject has a highly detectable immune response such that they did receive the vaccine. Accordingly, subjects that are not made aware of their placement in the treatment or placebo arm of a clinical trial could use the assays and methods described herein to determine whether or not they received the vaccine in the trial (e.g., vaccination status).

The assays and methods using the same scoring and stratification system described herein, may also be useful in providing a method for vaccinating the subject against the disease. Typically, the vaccine comprises an adjuvant as will be understood. The method comprises measuring disease-specific antibodies in the sample from the subject and determining the unit score based on the disease-specific antibodies measured. In this method, the unit score correlates to the immune status of the subject and based on that unit score, and the subject is vaccinated if the immune status has a predetermined unit score value. In typical aspects, the predetermined unit score value ranges from 0 to about 140, more typically, the predetermined unit score value ranges from 0 to about 90. In this way, the predetermined unit score value relates to the unit score values associated with, for example, the low immunity group unit scores described herein. As an example of this application, if the subject's unit score falls within the high immunity group, the unit score of the immune status of the subject would not be the predetermine unit score for vaccination and thus the subject would not be vaccinated. However, if the subject's unit score falls within the low immunity group, the unit score of the immune status of the subject would be the predetermined unit score value for vaccination and the subject would be vaccinated.

Additionally, the assays and methods using the same scoring and stratification system described herein, may also be useful in identifying whether a subject should be administered a particular treatment regime. For example, in respect of COVID-19 and the use of RNA vaccines, uncovering that a subject is in the high immunity group based on their unit score, prior to vaccination with said vaccine, could help limit health care costs both in respect of limiting wasteful use of the vaccine on individuals that don't require inoculation, but also in respect of limiting adverse effects of delivering the vaccine to said subject. In this respect, giving this subject the RNA vaccine would likely be detrimental to their health as this type of vaccine would likely result in the immune system overtly attacking the subject's own cellular system. In this way, a method for determining whether a subject should be administered an RNA vaccine against a disease may be provided. The method comprises measuring disease-specific antibodies in the sample from the subject and determining the unit score based on the disease-specific antibodies measured. In this method, the unit score predicts whether the subject should receive the RNA vaccine. Thus, the assays and methods described herein can be useful for avoidance of unnecessary and detrimental adverse effects of particular treatment regimes. Similarly, the methods described herein can be used to assess vaccine efficacy and determine, for example, if a particular subject may benefit from an additional dose of a vaccine or an alternate vaccine choice. In some aspects, the methods described herein include a step of obtaining a sample from the subject. In some aspects, the methods described herein include a step of administering an agent, such as a treatment, a vaccine, or a booster.

The probe used in the measurement of the disease-specific antibodies, typically comprises an antigen. This antigen can be any antigen of interest related to the disease in question, and is used to determine whether the humoral response (e.g., disease-specific antibodies), is detectable in the subject being tested. Typically, the antigen can be a protein, a peptide, a polypeptide, a carbohydrate or fragment thereof, that is recognized by antigen-specific antibodies in the subject's sample. Such recognition is used in the determination of the unit score. In most typical aspects, the antigen comprises SARS-CoV-2 S1 and S2 proteins or variants thereof. In typical aspects, the S1 and S2 proteins or variants adopt a substantially native conformation. In this way, the conformation of the epitope is biologically relevant and detects biologically relevant antibodies in the sample of the subject. In other aspects, the antigen comprises a spike protein conformational epitope or variant thereof, wherein the epitope is not present in S1 or S2 alone. In this aspect, the epitope is an epitope of the spike protein that is not a cryptic epitope. It is an epitope that is exposed in the formed spike protein, either alone or in trimeric form. It will be understood that variants include fragments as described herein and that the variants typically retain the native conformation of the peptide in question.

In additional or alternative aspects, the antigen comprises, consists essentially of, or consists of the polypeptide sequence of SEQ ID NO:1:

| | |
|---|---|
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfcndpf lgvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqdvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkfngltv lpplltdemi aqytsallag titsgwtfga gaalqipfam |
| 901 | qmayrfngig vtqnvlyenq klianqfnsa igkiqdslss tasalgklqd vvnqnaqaln |
| 961 | tlvkqlssnf gaissvlndi lsrldkveae vqidrlitgr lqslqtyvtq qliraaeira |
| 1021 | sanlaatkms ecvlgqskrv dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyeq |

The sequences may further comprise additional features, such as additional sequences as fusion proteins, stabilizing residues, such as proline residues, targeting sequences, or other such variations known to the skilled person to impact efficacy and/or potency for detecting and/or generating antibodies. For example, typically, a pair of proline residues are added at the C-terminus to improve stability. In other typical aspects, the trunk protein of SARS-CoV-2 is included in the sequence.

Typically, the variant comprises at least about 50%, at least about 60%, at least about 70%, at least about 80%, at least about 90%, at least about 95%, or at least about 99% sequence identity to the native S1 and S2 proteins, the native conformational epitope, or SEQ ID NO:1.

It will be understood that these proteins are typically glycosylated in vivo and the recombinant versions described herein may or may not be similarly glycosylated. Typically, however, the antigen is N-glycosylated. The antigen may be partially or fully N-glycosylated and it may be synthetically N-glycosylated or naturally N-glycosylated for example through production in mammalian cells. The antigen is, for example, N-glycosylated at about 50%, about 60%, about 70%, about 80%, about 90%, about 95%, or about 99%, or more, or 100% of the native N-glycosylation sites.

It will be understood that the S protein of SARS-CoV-2 is trimeric. The antigen thus is also typically trimeric in the diagnostic assays and/or vaccinations described herein. The antigen, however, can be in monomeric form or other oligomeric forms, provided the conformational epitope(s) remain in their substantially native conformation.

While the use of SARS-CoV-2 S1 and S2 proteins as the antigen of interest have been described and exemplified in detail, the assays and methods described herein are not to be limited to the use of this particular antigen or to only infectious diseases. In typical aspects, the determination of the immune status of the subject is related to disease of the subject. In this way, since the measurement and determination of the unit score is related to the measurement of disease-specific antibodies, the assays and methods described herein are related to the immunity the subject has in respect of any type of disease (e.g., infectious disease (which would be the case for SARS-CoV-2 and the S1 and S2 protein antigens described herein) or non-infectious disease).

Examples of infectious diseases applicable to the assays and methods described herein include, but are not limited to, hepatitis, strep throat, urinary tract infections, tuberculosis, malaria, dengue fever, meningococcal disease, cholera, rabies, ebola, influenza, coronavirus disease-2019 (COVID-19), herpes, respiratory infection, Severe Acute Respiratory Syndrome (SARS), Middle East Respiratory Syndrome (MERS), mumps, measles, rubella, polio, small pox, human immunodeficiency virus (HIV), acquired immune deficiency syndrome (AIDS), pneumonia, Lyme disease, anthrax, tetanus, cholera, plague, diphtheria, chlamydia or malaria. In most typical embodiments, when the disease is an infectious disease, the infectious disease is coronavirus disease-2019 (COVID-19). Moreover, when the disease is COVID-19, the antigen is SARS-CoV-2 and the S1 and S2 protein as described herein.

The antigen may be from another infectious agent such as, another virus (i.e., other than SARS-CoV-2), or be a completely different microorganism, such as a bacteria, protozoa, fungi and the like. Additionally, the antigen does not need to be limited to an infectious agent, but it may be a non-infectious agent, such as an autoantigen used in the detection of autoantibodies or a food antigen used for the detection of allergen-specific antibodies, in the subject's sample. Examples of non-infectious diseases applicable to the assays and methods described herein include, but are not limited to, cancer, allergic disease, cardiovascular disease, cardiac disease, a disease of the central nervous system, diabetes, autoimmune disorder, and a disorder associated with inflammation. The skilled person would understand that any antigen capable of detecting the humoral response in the subject, wherein the detection can be converted into the unit score described herein, can be used in the assays and methods described herein.

The assay described herein may be of any form that detects antibodies. Typically, the assay is an enzyme-linked immunosorbent assay (ELISA), a lateral flow assay, or a chemiluminescence immunoassay. The assay can detect any type of antibody, depending on the sample in question, but typically the assay detects IgG, IgM, IgE and/or IgA.

The sample tested in the assay may be any fluid, such as a bodily fluid, such as blood, blood plasma, blood serum, hemolysate, spinal fluid, urine, lymph, synovial fluid, saliva, sperm, amniotic fluid, lacrimal fluid, cyst fluid, sweat gland secretion, and bile. Typically, the sample is a blood sample.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Methods

### ELISA Assay:

SARS-COV-2 protein (S total recombinant) 2 ug/mL in PBS was coated on a microplate overnight at 4° C. Wells were blocked with 3% BSA in PBS with 1.0% Tween-20 (PBS-T) for 1 hour at 37° C. Next, wells were washed with PBS-T and incubated with 1:100 diluted sera in 3% BSA in PBS-T for 1 hour at room temperature under agitation. Wells were washed with PBS-T and incubated with 1:7500 dilution of horseradish peroxidase (HRP)-conjugated goat anti-human IgG antibody for 1 hour at room temperature under agitation. Then, wells were washed with PBS-T. Wells were developed using 3, 3′, 5, 5′; -tetramethylbenzidine for 15 minutes at dark and the stop solution was added (0.5 M H2SO4). The absorbance was read at 450 nm.

### Antigen Source:

S1—recombinant-Recombinant SARS-CoV-2 51 glycoprotein (NCBI accession number YP_009724390.1, AA1-674), C-terminally tagged with a predominantly monomeric sheep Fc-tag. Protein was produced in HEK293 cells and purified from culture supernatant by Protein G chromatography. The S1 protein was express on HEK293 mammalian cells to obtain more authentic post-translational modifications. (The Native Antigen Company-UK REC31806-500)

S total—The full-length spike protein sequence (S total antigen). GenBank:

MN908947. It was expressed ectodomain residues 1 to 1208 of 2019-nCoV S, adding two stabilizing proline mutations in the C-terminal, trimeric and stabilized version express in cells mammalian cells fully glycosylated S protein. (https://doi.org/10.1101/2020.03.17.20037713)

### Results

### Coating Optimization Using Recombinant S1 Protein

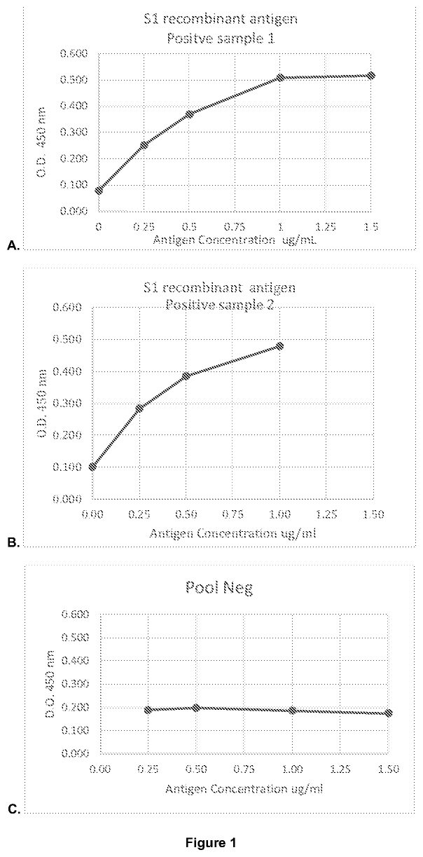

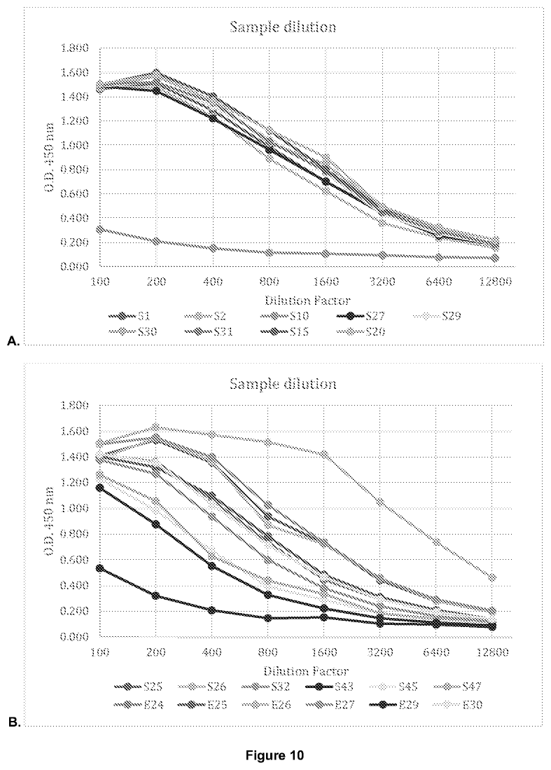

Coating optimization experiments were first carried out in order to determine how much S1 and S total to use in subsequence experiments. FIG. 1A shows the dose response for S1 antigen concentration used for positive sample 1, FIG. 1B shows the dose response for S1 antigen concentration used for positive sample 2, and FIG. 10 shows the dose response for S1 antigen concentration using a pool of negative samples.

### Antigen Comparison

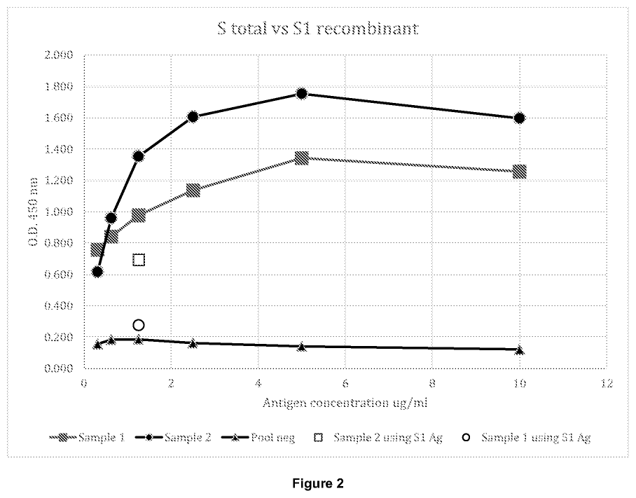

Dose response curves were compared using S total and S1 antigens on two different positive samples and a pool of negative samples (FIG. 2). Shaded circles represent S total tested on sample 1 at a variety of concentrations, shaded squares represent S total tested on sample 2 at a variety of concentrations, and shaded triangles represent S total tested on a pool of negative samples at a variety of concentrations. The non-shaded circle and square represent S1 tested on samples 1 and 2, respectively, at 0.5 ug/ml. It is evident that S total was better able to reflect a positive result at a lower concentration than S1 in both of these samples.

### S Total Coating Concentration

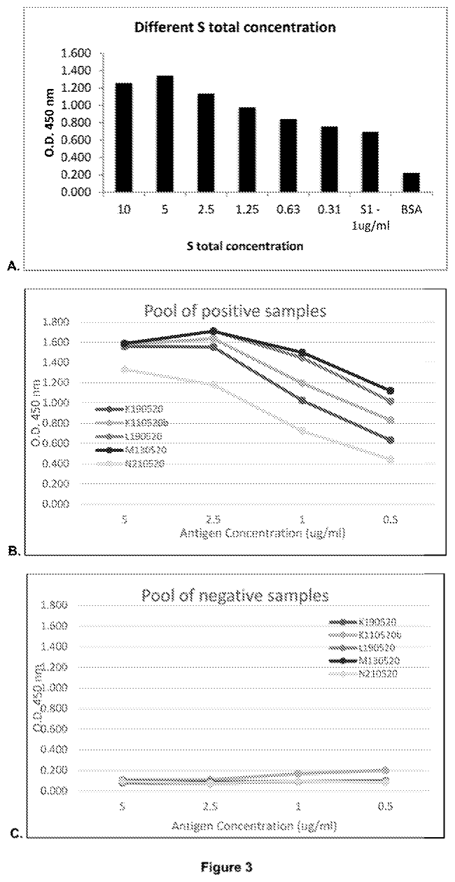

The coating of S total protein was evaluated at different concentrations in order to establish parameters for subsequent testing. FIG. 3A shows results using various concentrations of S total protein as compared to S1 at 1 ug/ml and a BSA control. FIG. 3B shows the results of S total at various concentrations using a pool of positive samples and FIG. 3C shows the results of S total at various concentrations using a pool of negative samples.

### Concentration of Secondary Antibody

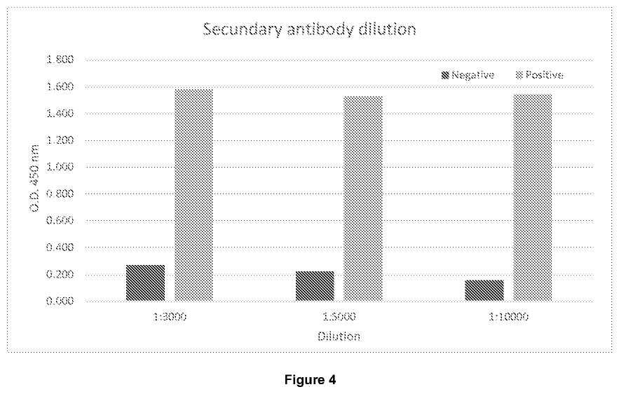

FIG. 4 shows the results of testing to determine appropriate amounts of secondary antibody to use in subsequent testing.

### Cut-Off Determination

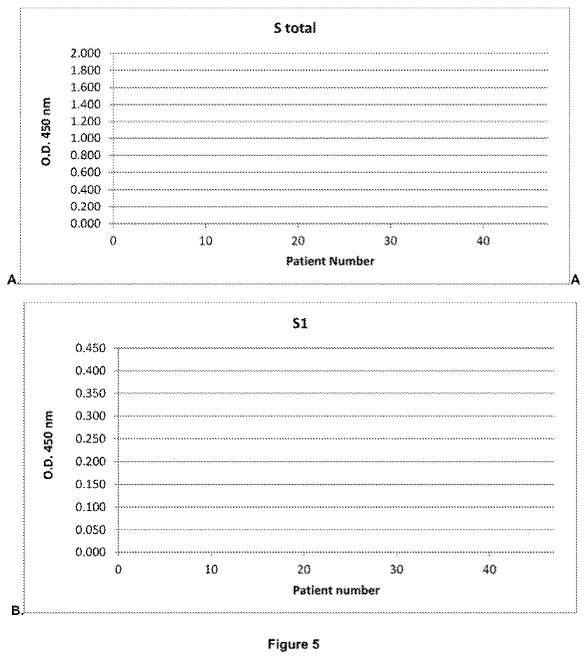

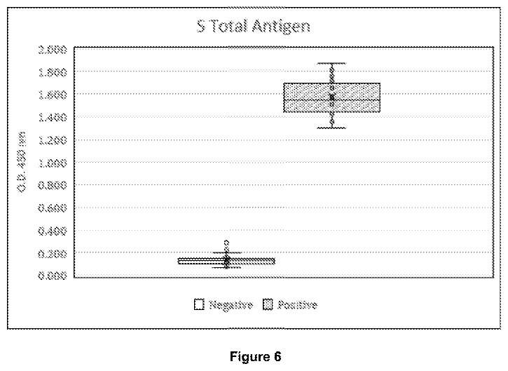

Cutoff determination assays were performed using S1 and S total. Assays were performed as before. Plates were coated with S1 (0.5 ug/ml at 50 ul per well) or S total (2 ug/ml at 50 ul per well) using the same incubation times and concentrations. FIG. 5A shows the results with S total and FIG. 5B shows the results with S1. When S total is used, there is a clear separation between positive and negative samples. When S1 is used, the separation between positive and negative samples is unclear and there appear to be some false positive and some false negative results. FIG. 6 shows the comparison between positive and negative samples using S total as the antigen. The different between positive and negative samples is statistically significant at a p value of <0.000001.

### Improving Background Signal

Different BSA concentrations were tested in order to improve the background signal. Results are shown in Table 1:

TABLE 1

| | BSA 3% | BSA 4% | BSA 5% |
|---|---|---|---|
| Positive Control | 1.449 | 1.450 | 1.455 |
| Neg1 | 0.349 | 0.332 | 0.367 |
| Neg2 | 0.468 | 0.404 | 0.382 |
| Neg3 | 0.281 | 0.317 | 0.296 |
| Neg4 | 0.425 | 0.515 | 0.435 |
| Neg5 | 0.335 | 0.353 | 0.362 |
| Neg6 | 0.354 | 0.308 | 0.321 |
| Neg7 | 0.488 | 0.565 | 0.531 |
| Neg8 | 0.578 | 0.478 | 0.537 |
| Neg9 | 0.427 | 0.39 | 0.408 |
| Neg10 | 0.242 | 0.239 | 0.232 |
| Neg11 | 0.331 | 0.376 | 0.333 |
| OD average | 0.389 | 0.389 | 0.382 |
| OD SD | 0.098 | 0.097 | 0.093 |

### Improving Differences Between Positive and Negative Samples

Different Tween concentrations were tested in order to improve the differences observed between positive and negative samples. Results are shown in Table 2:

TABLE 2

| | BSA 3% pool | BSA 3% pool | BSA 3% pool |
|---|---|---|---|
| | liq. + PBS-T | liq. + PBS-T | liq. + PBS-T |
| | 0.05% | 0.1% | 1% |
| Positive Control | 1.357 | 1.494 | 1.472 |
| Neg1 | 0.254 | 0.264 | 0.228 |
| Neg2 | 0.419 | 0.323 | 0.276 |
| Neg3 | 0.279 | 0.188 | 0.195 |
| Neg4 | 0.449 | 0.369 | 0.305 |
| Neg5 | 0.335 | 0.273 | 0.23 |
| Neg6 | 0.167 | 0.232 | 0.229 |
| Neg7 | 0.550 | 0.444 | 0.406 |
| Neg8 | 0.450 | 0.366 | 0.327 |
| Neg9 | 0.416 | 0.29 | 0.289 |
| Neg10 | 0.227 | 0.196 | 0.178 |
| Neg11 | 0.350 | 0.288 | 0.177 |
| OD average | 0.354 | 0.294 | 0.258 |
| negative | | | |
| OD SD | 0.115 | 0.078 | 0.070 |

### Patient Sample Stability

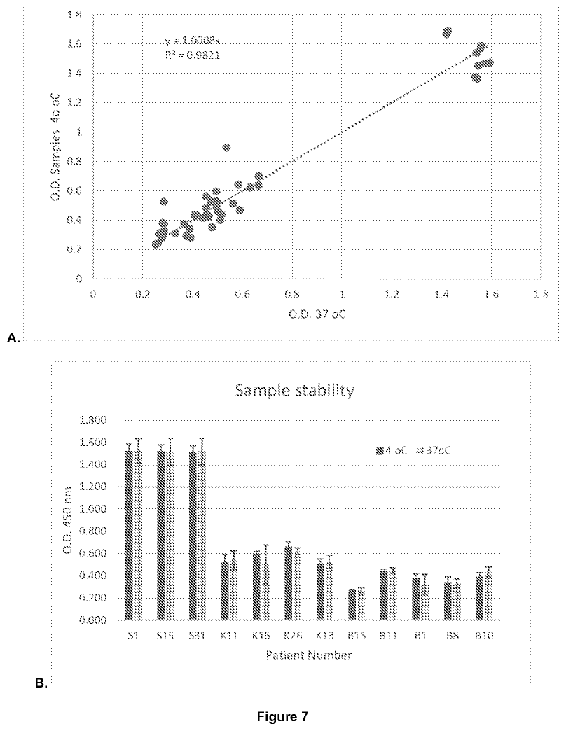

To confirm patient sample stability, a number of samples were tested after 7 days at 4° C. or 37° C. The samples were found to have similar stabilities at each temperature, as shown in Table 3 and in FIGS. 7A and 7B:

TABLE 3

| 7 days | 4° C. | 37° C. |
|---|---|---|
| Sample 1 | 1.419 | 1.667 |
| Sample 2 | 1.594 | 1.475 |
| Sample 3 | 1.558 | 1.574 |
| Sample 4 | 1.536 | 1.376 |
| Sample 5 | 1.421 | 1.683 |
| Sample 6 | 1.548 | 1.454 |
| Sample 7 | 1.56 | 1.588 |
| Sample 8 | 1.538 | 1.367 |
| Sample 9 | 1.426 | 1.69 |
| Sample 10 | 1.571 | 1.469 |
| Sample 11 | 1.54 | 1.539 |
| Sample 12 | 1.543 | 1.365 |
| Sample 13 | 0.631 | 0.625 |
| Sample 14 | 0.563 | 0.515 |
| Sample 15 | 0.455 | 0.563 |
| Sample 16 | 0.511 | 0.402 |
| Sample 17 | 0.474 | 0.528 |
| Sample 18 | 0.495 | 0.501 |
| Sample 19 | 0.501 | 0.464 |
| Sample 20 | 0.537 | 0.894 |
| Sample 21 | 0.665 | 0.636 |
| Sample 22 | 0.583 | 0.644 |
| Sample 23 | 0.666 | 0.703 |
| Sample 24 | 0.59 | 0.471 |
| Sample 25 | 0.495 | 0.597 |
| Sample 26 | 0.497 | 0.53 |
| Sample 27 | 0.517 | 0.441 |
| Sample 28 | 0.26 | 0.246 |
| Sample 29 | 0.265 | 0.31 |
| Sample 30 | 0.278 | 0.283 |
| Sample 31 | 0.253 | 0.237 |
| Sample 32 | 0.466 | 0.427 |
| Sample 33 | 0.454 | 0.483 |
| Sample 34 | 0.441 | 0.418 |
| Sample 35 | 0.417 | 0.433 |
| Sample 36 | 0.376 | 0.293 |
| Sample 37 | 0.285 | 0.378 |
| Sample 38 | 0.33 | 0.311 |
| Sample 39 | 0.286 | 0.527 |
| Sample 40 | 0.392 | 0.28 |
| Sample 41 | 0.286 | 0.326 |
| Sample 42 | 0.366 | 0.377 |
| Sample 43 | 0.28 | 0.383 |
| Sample 44 | 0.408 | 0.438 |
| Sample 45 | 0.479 | 0.352 |
| Sample 46 | 0.461 | 0.434 |
| Sample 47 | 0.389 | 0.34 |
| | 4° C. Average | 37° C. Average |
| P = 0.596 NS | 0.715 | 0.724 |

### Determination of Coefficient of Variation for S Total

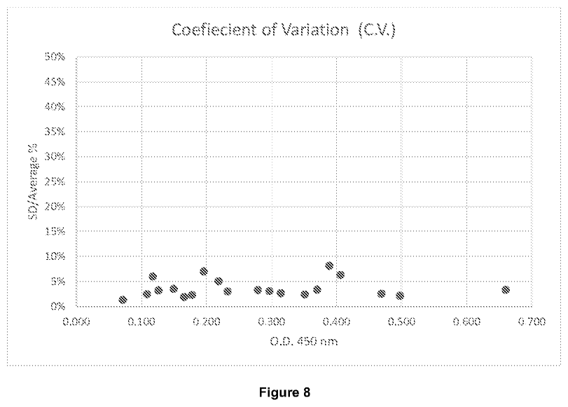

The coefficient of variation was determined for S total antigen as shown in FIG. 8. Each sample was run in triplicate using the average and SD of each point.

### Determination of Intrassay Variation

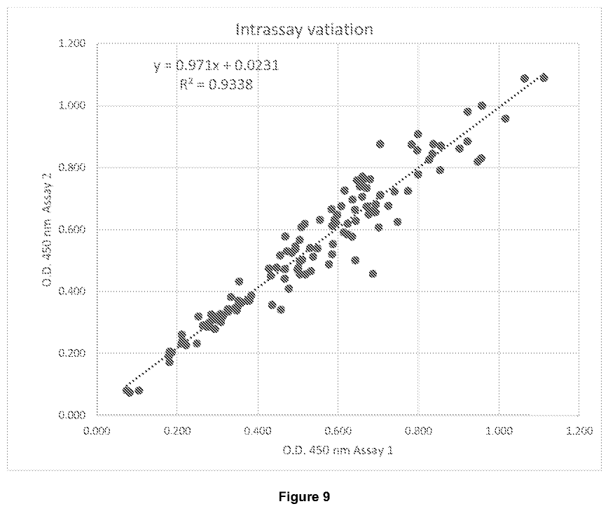

Assays done using separate plates and operators were correlated and found to have no significant variation, as shown in FIG. 9.

### Determination of Assay Sensitivity and Specificity

Assay sensitivity and specificity was measured as shown in Table 4. Assay sensitivity is 98.21% and the Specificity is 98.80%. The calculate Positive Predicted Value (PPV) is 96.49% and the Negative Predicted Value is 99.39%.

TABLE 4

| | | COVID19 | Pre-COVID19 | |
|---|---|---|---|---|
| TEST | | Positive Samples | Samples | Total |
| | POSITIVE | 55 | 2 | 57 |
| | NEGATIVE | 1 | 165 | 166 |

| | | | |
|---|---|---|---|
| Total | 56 | 167 | 223 |

### Sample Titration

Different positive samples were sequentially diluted from 1:100 up to 1:12800. The assay was performed as described previously. Result are shown in FIGS. 10A and 10B, where each line represents a different patient sample.

### Immunity Evaluation of Patient Family Clusters

FIG. 11 shows assay results for Patient Cluster 1 using S total antigen. The horizontal line represents the assay cutoff indicating a positive result. Patient A was hospitalized due to SARS-COV 2 infection. The patient's spouse and family member living in the same house did not develop COVID2 symptoms but both tested positive.

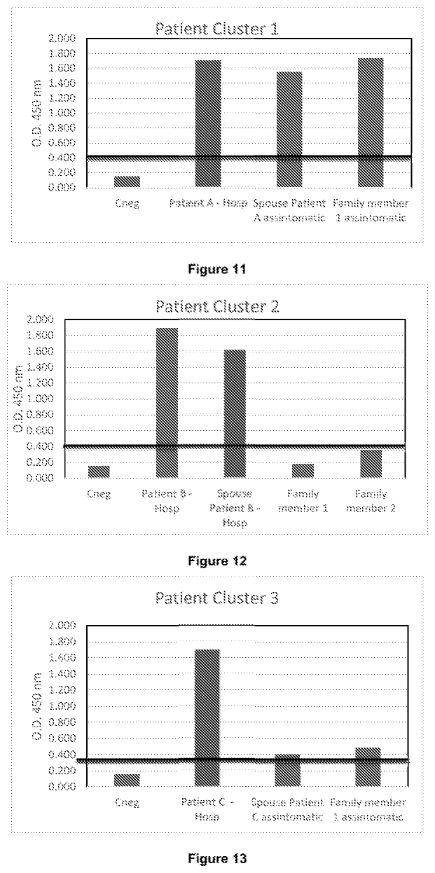

FIG. 12 shows assay results for Patient Cluster 2 using S total antigen. The horizontal line represents the assay cutoff indicating a positive result. Patient B was hospitalized due to SARS-COV 2 infection. The patient's spouse also developed COVID2 and was also hospitalized two days apart. Neither family members 1 nor 2 developed COVID2 symptoms and both tested negative.

FIG. 13 shows assay results for Patient Cluster 3 using S total antigen. The horizontal line represents the assay cutoff indicating a positive result. Patient C was hospitalized due to SARS-COV 2 infection. The patient's spouse and family member living in the same house did not develop COVID2 symptoms but both tested positive.

### Immunity Evaluation in a Company Setting

Samples were collected different times from a group of co-workers at a medical clinic. The samples were kept at −20° C. and assayed all together in the same assay using S total antigen. Results are shown in

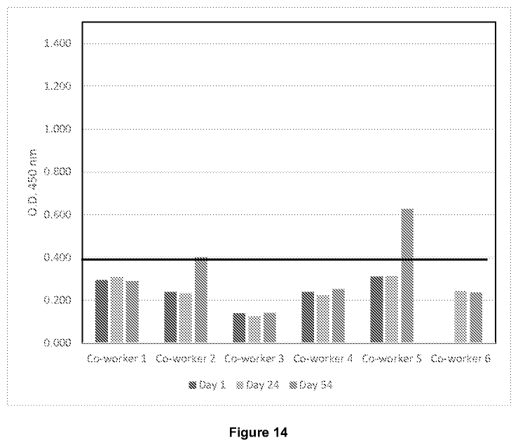

FIG. 14. The horizontal line represents the assay cut-off. From these results, it is clear that by day 54, both co-workers 1 and 5 had positive test results.

### Comparison Between S1 and S Total

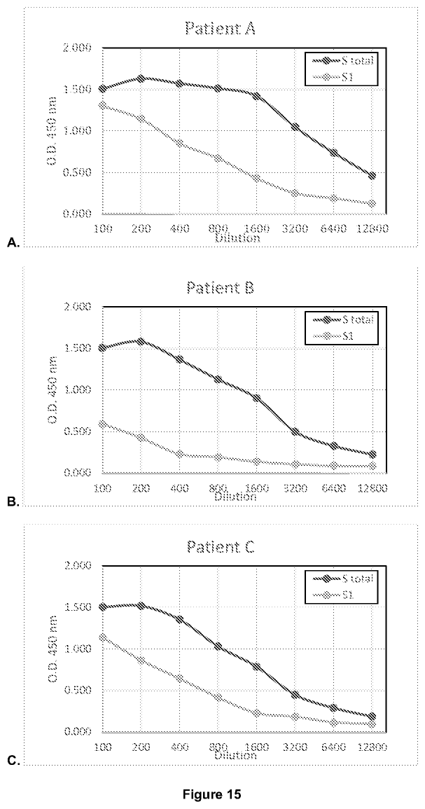

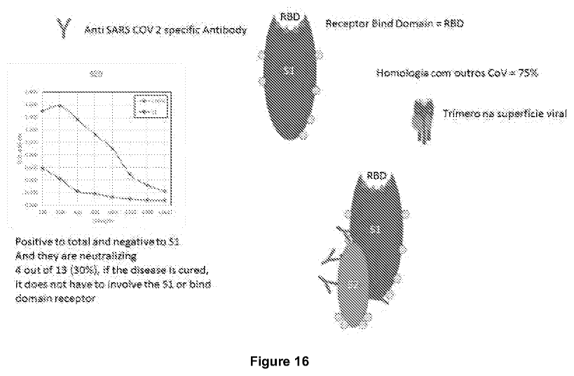

S1 and S total were compared across a number of different patients and it was identified that in many cases, the patient's COVID2 result would be deemed negative if S1 was used and positive if S total was used, depending on the dilution factor. FIGS. 15A, 15B, and 15C show three exemplary results in this case. FIG. 16 shows schematically what is believed to be happening in this scenario. It is believed that the subjects showing a negative result to S1 but positive to S total have a higher proportion of antibodies that are specific for conformational epitopes formed by S1 and S2 together or are specific for S2. These antibodies would not be detected in an assay using only S1 as the antigen. The antibodies being detected in this case would likely be neutralizing antibodies.

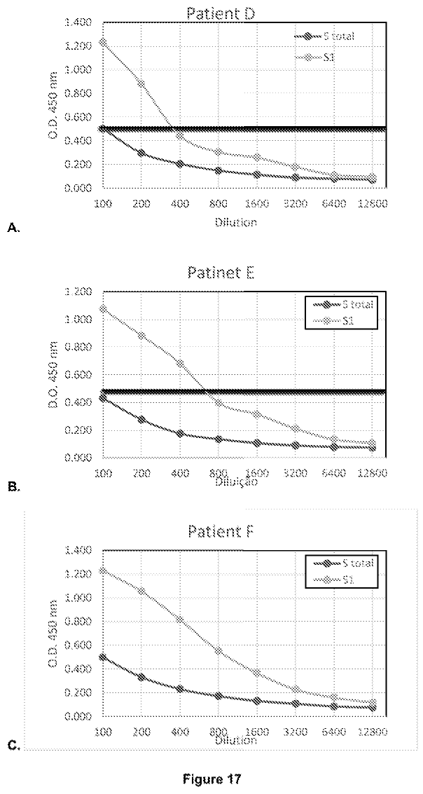

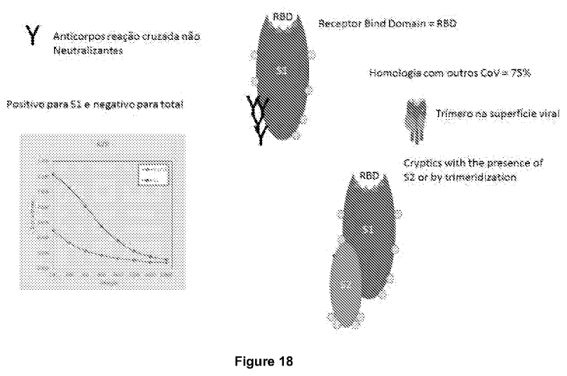

In other cases, the patient's COVID2 result would be deemed positive if S1 was used and negative if S total was used, as shown in FIGS. 17A, 17B, and 17C. FIG. 18 shows schematically what is believed to be happening in this scenario. It is believed that the subjects showing a positive result to S1 but negative to S total have cross-reactive antibodies that are specific for cryptic epitopes of S1 that would not be exposed in the full spike protein. These antibodies would not be detected in an assay using only S total as the antigen. The antibodies being detected in this case would not likely be neutralizing antibodies.

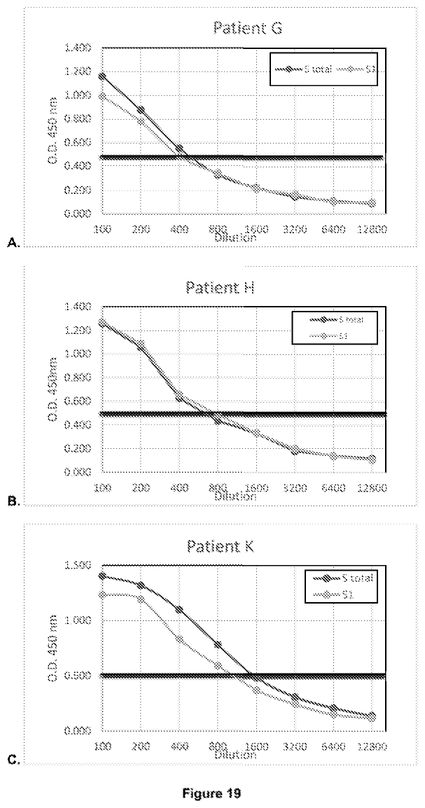

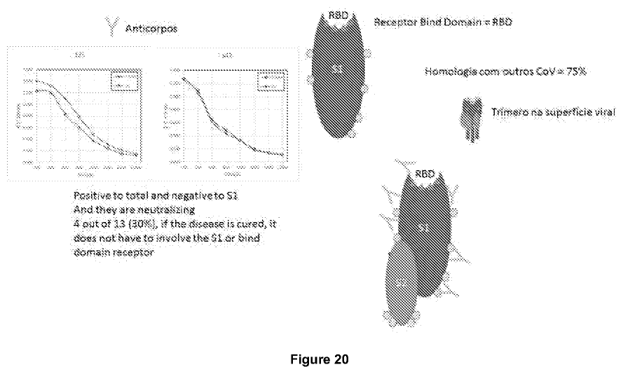

In still other cases, the results using S1 and S total would be similar and closely tracked each other, as shown in FIGS. 19A, 19B, and 19C. FIG. 20 shows schematically what is believed to be happening in this scenario. It is believed that the subjects showing positive results both S1 and S total have antibodies that are specific for epitopes of S1 that would be exposed in the full spike protein. These antibodies would be detected in an assay using either S1 or S total as the antigen. The antibodies being detected in this case would likely be neutralizing antibodies.

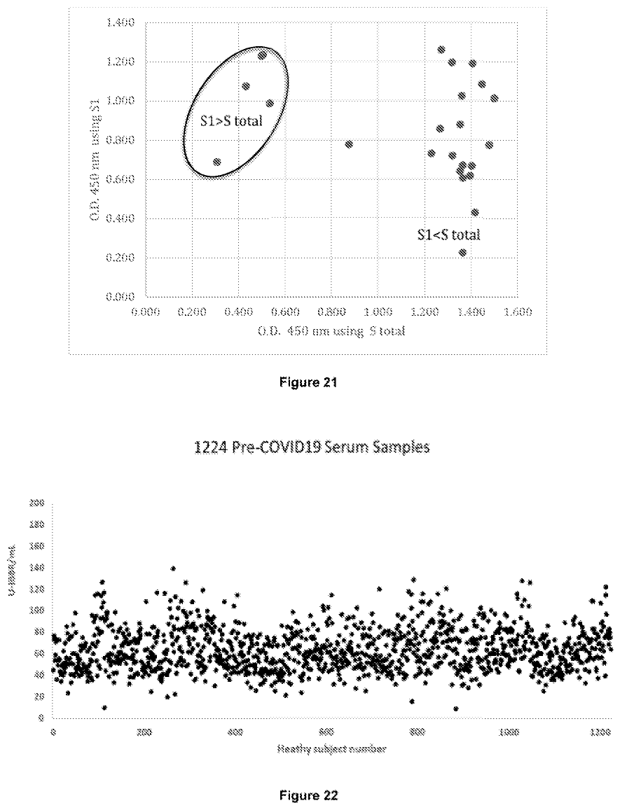

As shown in FIG. 21, the O.D. between S total and S1 was compared for a number of patients. It can be seen that most patients tested positive for S1 and S total. There were, however, some patients that tested positive for S1 and negative for S total (these would be false positive results if S1 was used and represent antibodies that will not bind to S total and would not provide immune protection against SARS-CoV-2) and some patients that tested negative for S1 and positive for S total (these would be false negative results if S1 was used and represent patients that have immunity against SARS-CoV-2 but would not be identified using S1 alone).

### Patient Comparison Using S1 or S Total

Table 5 shows patient results using both S total and S1 as the antigen in the assay. Clinical confirmation of COVID19 was used to confirm a positive result. From this table, it can be seen that all 19 patients that clinically developed COVID19 were consider positive using the S total antigen. 4 out of 19 patients (21%) were considered negative using an assay with the S1 antigen. This means that approximately 20% of COVID19 patients developed immunity independently of S1 epitopes. Either these patients developed an immune response against S2 epitopes, conformational epitopes or carbohydrate containing epitopes that are not present on the S1 antigen.

TABLE 5

| | | O.D. S | 0.D. | | |
|---|---|---|---|---|---|
| Clinical Data | Patient # | Total | S1 | S total | S1 |
| Without COVID19 history | PK16 | 0.431 | 1.073 | Negative | Positive |
| Without COVID19 history | PK26 | 0.498 | 1.228 | Negative | Positive |
| Without COVID19 history | PK11 | 0.504 | 1.233 | Negative | Positive |
| Without COVID19 history | PS10 | 0.307 | 0.687 | Negative | Positive |
| Without COVID19 history | PS43 | 0.534 | 0.986 | Negative | Positive |
| Clinical COVID19 | PS47 | 1.272 | 1.259 | Positive | Positive |
| Clinical COVID19 | PE25 | 1.318 | 1.193 | Positive | Positive |
| Clinical COVID19 | PE29 | 0.875 | 0.776 | Positive | Positive |
| Clinical COVID19 | PE24 | 1.360 | 1.024 | Positive | Positive |
| Clinical COVID19 | PE27 | 1.267 | 0.856 | Positive | Positive |
| Clinical COVID19 | PS01 | 1.500 | 1.013 | Positive | Positive |
| Clinical COVID19 | PE30 | 1.353 | 0.877 | Positive | Positive |
| Clinical COVID19 | PS45 | 1.230 | 0.730 | Positive | Positive |
| Clinical COVID19 | PS02 | 1.478 | 0.774 | Positive | Positive |
| Clinical COVID19 | PS31 | 1.353 | 0.640 | Positive | Positive |
| Clinical COVID19 | PS32 | 1.396 | 0.616 | Positive | Negative |
| Clinical COVID19 | PS29 | 1.319 | 0.719 | Positive | Positive |
| Clinical COVID19 | PS26 | 1.364 | 0.670 | Positive | Positive |
| Clinical COVID19 | PS25 | 1.407 | 1.188 | Positive | Positive |
| Clinical COVID19 | PS27 | 1.446 | 1.083 | Positive | Positive |
| Clinical COVID19 | PS15 | 1.403 | 0.666 | Positive | Positive |
| Clinical COVID19 | PS30 | 1.365 | 0.605 | Positive | Negative |
| Clinical COVID19 | PE26 | 1.417 | 0.427 | Positive | Negative |
| Clinical COVID19 | PS20 | 1.364 | 0.225 | Positive | Negative |
| | | | Cut-off | 0.613 | 0.657 |
| | | | Neg Control | 0.163 | 0.207 |

It is interesting to note that all patients without a COVID19 clinical history that were positive using S1 antigen assay were negative when S total antigen was used. This may represent cross reactive antibodies that do not mediate neutralization, thereby surrendering a false positive immunity when the S1 antigen was used.

### Example 2

### Unit Score for Healthy (Pre-COVID-19) Patients

As shown in FIG. 22, serum samples from 1224 healthy patients (serum samples were taken from 2016 to 2018; no known exposure to COVID-19) were taken for analysis and determination of SARS-CoV-2 antibody levels. The antibody levels were used to develop an arbitrary unit score (U-IBBR), based on the pool of COVID-19 convalescent patients—this pool of serum was established to contain 200 U-IBBR. The data was normalized using understood statistical methods.

### Distribution of the Data from Healthy (Pre-COVID-19) Patients

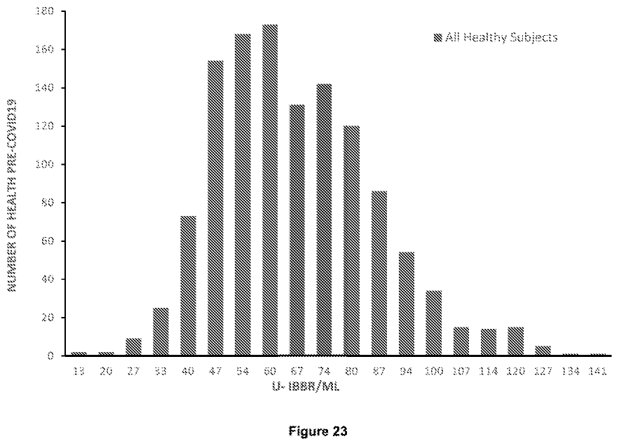

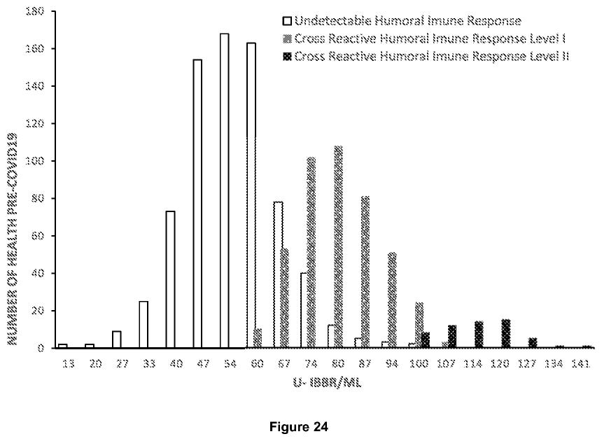

As shown in FIG. 23, a distribution curve, illustrating the spread of the data, was produced from the data collected from the 1224 samples. The curve does not have a normal distribution, because, as shown in FIG. 24, the distribution curve was determined to be the integration of three different curves, 1) undetectable humoral response group (60.15% of the samples), 2) humoral response with cross reactivity level I (35.27% of the samples), and 3) humoral response with cross reactivity level II (4.58% of the samples). These groups correlated with the unit score (U-IBBR), as shown along the x axis, where group 1 had an U-IBBR of 0 to about 50, group 2 had an U-IBBR of about S1 to about 100 and group 3 had an U-IBBR of about 101 to about 140.

### Unit Score for Positive COVID-19 (Recovered) Patients

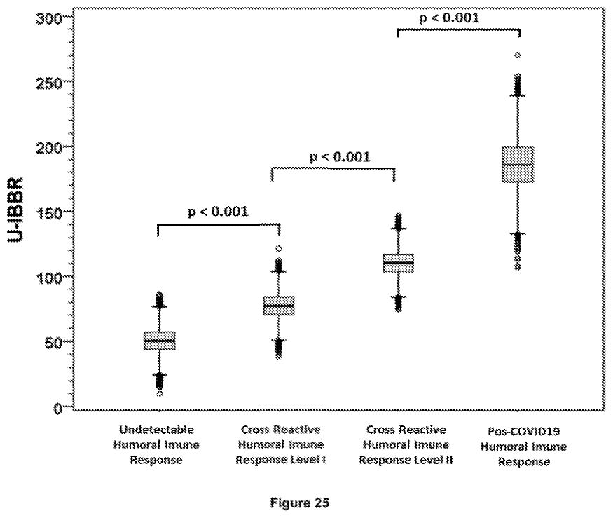

As shown in FIG. 25, serum from 700 patients who recovered from COVID-19 (e.g., previously positive for COVID-19) “Pos-COVID19” was analyzed for SARS-CoV-2 antibody levels using the methods described above. A fourth population or group (in addition to the three described above) was detected. This group had its own unit score (U-IBBR), as shown in the y axis of about 200. The differences between the four groups, based on their unit score (U-IBBR) was statistically significant.

### Algorithm and Prediction of Immune Status/Profile of a Subject

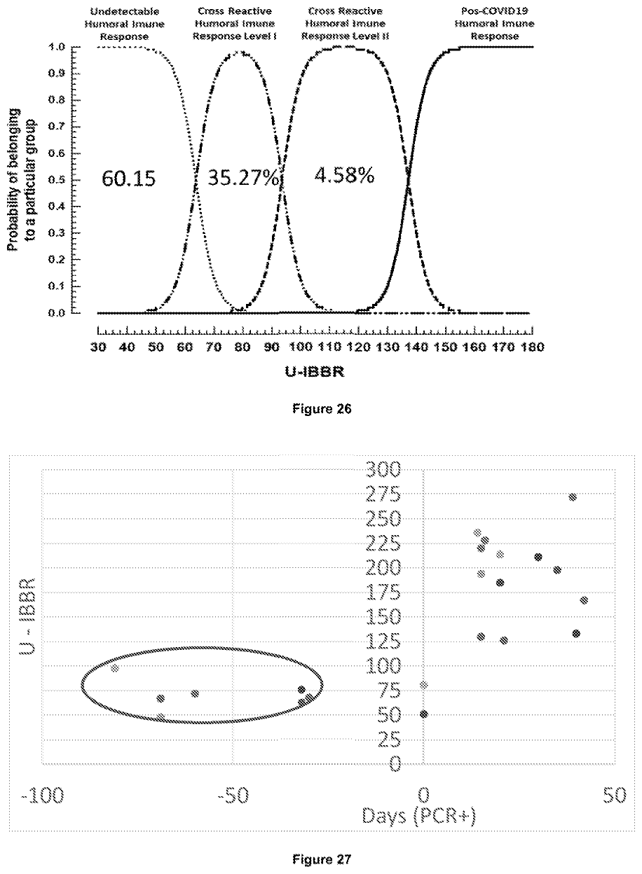

As shown in FIG. 26, the probability of belonging to a particular group (i.e. 1, 2, 3 or 4) was determined using an algorithm incorporating the unit scores determined above. Briefly, after describing the U-IBBR distribution, four sub-populations were assumed with mean±SD of 50±10, 77±10, 110±10, and 186±20, respectively. Computer simulations generated 10,000 normally distributed replications for each sub-population, creating 40,000 observations using the rnorm( )function of the R program. To assess normality we used the Jarque-Bera test. An ordinal logistic regression model was fit to the data and the probability of belonging to each sub-population was calculated for each observation. Group probabilities were then plotted against U-IDBR values. Data were analyzed using SPSS version 22.0 and R version 4.0. Accordingly, depending on what the tested subject's unit score is, the subject is placed into one of the aforementioned four groups, with the first group having no and/or low immunity (e.g., the patient is not immune to the virus) and the fourth group having high immunity (e.g., a subject in fourth group has been exposed to the virus (whether symptomatic or not) and is now immune to infection). It was found that patients with an unit score of about 200 U IBBR have antibody levels equivalent to those in the pool of cured COVID-19 patients.

### Quantitative Assessment (Level of Immune Response):

1) In addition to reagent, indeterminate or non-reagent result in the assay, a reactivity index was produced taking into account the optical density (O.D.) of the positive control (PC) standard containing 200 U IBBR.

TABLE 6
Example: Positive Control (PC)

| Wells containing positive control serum | Optical Density |
|---|---|
| (PC) | (O.D.) |
| C1 | 1.435 |
| D1 | 1.417 |
| Total | 2.852 |

Average O.D. for the positive control (PC) = O.D. total ÷ 2
Average = 2.852 ÷ 2 = 1.426

2) The reactivity index: the positivity index (titration): To also indicate a positivity index, a R positivity index was defined in each patient's result, which was calculated using the following formula:

Index R=O.D. of the patient/(O.D. N.C.+0.350)−Reagent (positive)

TABLE 7

| | | Index R | |
|---|---|---|---|
| Patient Serum | O.D. | Patient O.D./(O.D. NC + 0.350) | Index R |
| PC (média) | 1.426 | 1.426/(0.115 + 0.350) | 3.06 |
| F1 | 0.479 | 0.476/(0.115 + 0.350) | 1.03 |
| G4 | 1.062 | 1.062/(0.115 + 0.350) | 2.28 |
| H5 | 0.825 | 0.825/(0.115 + 0.350) | 1.77 |
| F5 | 2.022 | 2.022/(0.115 + 0.350) | 4.35 |

As shown in Table 8, determining a subject's unit score U-IBBR is predictive of their immune status (e.g. it is possible to calculate the chance of being in one of the aforementioned groups). For example, based on the data produced by the algorithm (Table 8), a subject that has a unit score of 30 is 100% likely/predicted to be in group 1 (undetectable humoral response and therefore has low or no immunity to the virus), a subject with a unit score of 80 is 97.60% likely to be in group 2 (humoral response cross reactive level I) and only 1.66% likely to be in group 3 (humoral response cross reactive group II), a subject with a unit score of 130 is 89.62% likely to be in group 3 (humoral response cross reactive level II) and 10.38% likely to be group 4 (pos-COVID-19 humoral response), whereas a subject with a unit score of 180 or better is 100% likely/predicted to be in group 4 (pos-COVID-19 humoral response).

TABLE 8

| | Undetectable | Cross Reactive | Cross Reactive | Pos-COVID19 |
|---|---|---|---|---|
| | Humoral Imune | Humoral Imune | Humoral Imune | Humoral Imune |
| | Response | Response Level I | Response Level II | Response |
| 30 U-IBBR | 100.00% | 0.00% | 0.00% | 0.00% |
| 40 U-IBBR | 99.92% | 0.08% | 0.00% | 0.00% |
| 50 U-IBBR | 98.43% | 1.57% | 0.00% | 0.00% |
| 60 U-IBBR | 75.52% | 24.48% | 0.00% | 0.00% |
| 70 U-IBBR | 13.21% | 86.71% | 0.08% | 0.00% |
| 80 U-IBBR | 0.74% | 97.60% | 1.66% | 0.00% |
| 90 U-IBBR | 0.04% | 74.51% | 25.46% | 0.00% |
| 100 U-IBBR | 0.00% | 12.60% | 87.40% | 0.00% |
| 110 U-IBBR | 0.00% | 0.71% | 99.26% | 0.03% |
| 120 U-IBBR | 0.00% | 0.04% | 99.40% | 0.57% |
| 130 U-IBBR | 0.00% | 0.00% | 89.62% | 10.38% |
| 140 U-IBBR | 0.00% | 0.00% | 29.78% | 70.22% |
| 150 U-IBBR | 0.00% | 0.00% | 2.05% | 97.95% |
| 160 U-IBBR | 0.00% | 0.00% | 0.10% | 99.90% |
| 170 U-IBBR | 0.00% | 0.00% | 0.01% | 99.99% |
| 180 U-IBBR | 0.00% | 0.00% | 0.00% | 100.00% |
| 190 U-IBBR | 0.00% | 0.00% | 0.00% | 100.00% |
| 200 U-IBBR | 0.00% | 0.00% | 0.00% | 100.00% |

### Use of Unit Score to Determine Level of Protection Against Subsequent Infection

As shown in FIG. 27, serum samples from seven healthy patients (left side of the graph) were assayed and unit scores were determined as described above. These samples were obtained between 1 month and up to 80 days prior to COVID-19 infection. When the same seven patients were assessed prior to COVID-19 infection, the unit score (U-IBBR) value was found to increase, in some cases by 2-fold. Thus the results of this experiment show that while it is currently unclear what U-IBBR value offers protection against infection, values that are not associated with protection, for example, U-IBBR values of about 90 or less, do not appear to protect against subsequent infection of the subject. However, the subjects that were subsequently infected appear to have increase U-IBBR values when their sample was tested again (right side of the graph). Accordingly, a higher U-IBBR value may be indicative of a change in the immune status of the subject such that the subject is immune from the disease.

### Example 3

Table 9 shows measured IBBR values in samples from subjects prior to the COVID-19 era. In other words, these subjects could not have previously been exposed to SARS-CoV-2. Also shown are measured IBBR values in samples from subjects who had experienced a positive COVID-19 PCT test result and subsequently recovered. From this data, it can be seen that all tested naïve subjects had measured IBBR values that were below the threshold for group 4 (pos-COVID-19 humoral response) suggesting that this group of subjects had very little natural immunity to SARS-CoV-2. Further, in subjects that had tested positive for COVID-19, all had measured IBBR values that were above the threshold to indicate that they would be in group 3 (humoral response cross reactive level II) and group 4 (pos-COVID-19 humoral response), suggesting that most had developed substantial immunity to SARS-CoV-2.

TABLE 9

| | | Samples collected | Samples collected |
|---|---|---|---|
| | | 2015-2018 | March-April 2020 |
| | | Pre-COVID Era | pos-COVID (cure) |
| ≤63 | Undetectable | 60% | 0% |
| 64 to 93 | Cross reactivity | 35% | 0% |
| | level I | | |
| 94 to 136 | Cross reactivity | 5% | 3% |
| | level II | | |
| ≥137 | Pos-Covid | 0% | 98% |
| | average U IBBR | 63 | 180 |
| | SD U IBBR | 19 | 20 |
| | | N = 1267 | N = 847 |
| | | | p < 0.0001 |

Table 10 shows measured IBBR values in non-vaccinated subjects who had previously had a positive or negative COVID-19 PCR result. In those who had received a positive PCR result, most fell into groups 3 or 4, suggesting high immunity to SARS-CoV-2. Those with lower immunity may have been tested early in their disease process and had not yet developed immunity to the disease. In those who had tested negative, most fell into groups 1 and 2, suggesting lower immunity or no immunity to SARS-CoV-2. Those with higher immunity may have experienced a previous infection for which they were not tested due to lack of testing availability or asymptomatic disease and thus had developed immunity prior to their negative test result.

TABLE 10

Samples April-June 2021 -
No vaccine
PCR (COVID19 by PCR)

| | | Positive | Negative |
|---|---|---|---|
| ≤63 | Undetectable | 3% | 35% |
| 64 to 93 | Cross reactivity | 7% | 27% |
| | level I | | |
| 94 to 136 | Cross reactivity | 17% | 16% |
| | level II | | |
| ≥137 | Pos-Covid | 73% | 22% |
| | average U IBBR | 190 | 100 |
| | SD U IBBR | 79 | 61 |
| | | N = 354 | N = 134 |
| | | | p < 0.0001 |

Table 11 shows measured IBBR values in non-vaccinated subjects who had previously had a positive or negative COVID-19 symptoms. In those who had COVID-19 symptoms, most fell into group 4, suggesting high immunity to SARS-CoV-2. Those with lower immunity may have been suffering from symptoms of a disease that is not COVID-19, such as the common cold. In those who had tested negative, most fell into either groups 1 or 4. Those with low immunity likely were truly not infected with SARS-COV-2, whereas those with higher immunity likely had asymptomatic disease.

TABLE 11

| | | Samples April-June 2021 - No vaccine | |
|---|---|---|---|
| | | Symptoms (COVID19 by symptoms) | |
| | | Positive | Negative |
| ≤63 | Undetectable | 11% | 29% |
| 64 to 93 | Cross reactivity | 13% | 17% |
| | level I | | |
| 94 to 136 | Cross reactivity | 17% | 17% |
| | level II | | |
| ≥137 | Pos-Covid | 60% | 37% |
| | average U IBBR | 166 | 121 |
| | SD U IBBR | 82 | 69 |
| | | N = 537 | N = 552 |
| | | | p < 0.0001 |

Table 12 shows measured IBBR values in non-vaccinated subjects who had no COVID-19 symptoms, stratified by their PCR test results. In asymptomatic patients with a positive PCR result, most had high immunity to COVID-19. In asymptomatic patients with a negative PCT result, most had low immunity to COVID-19. Those without a test done showed about equal results between low and high immunity.

TABLE 12

| | | Samples April-June 2021 - No vaccine | | |
|---|---|---|---|---|
| | | Symptoms negative (no COVID19 | | |
| | | by symptoms) | | |
| | | PCR | PCR | PCR Not |
| | | Positive | Negative | Done (N.D.) |
| ≤63 | Undetectable | 6% | 40% | 30% |
| 64 to 93 | Cross reactivity | 8% | 24% | 18% |
| | level I | | | |
| 94 to 136 | Cross reactivity | 20% | 13% | 18% |
| | level II | | | |
| ≥137 | Pos-Covid | 67% | 23% | 35% |
| | average U IBBR | 182 | 101 | 117 |
| | SD U IBBR | 81 | 65 | 65 |
| | | N = 51 | N = 62 | N = 439 |

Table 13 shows measured IBBR values in non-vaccinated subjects who had positive COVID-19 symptoms, stratified by their PCR test results. In symptomatic patients with a positive PCR result, most had high immunity to COVID-19. Those with lower immunity were likely tested too early in the disease process to have developed significant immunity. In symptomatic patients with a negative PCT result, most had low immunity to COVID-19. Those with higher immunity likely had previous disease. Those without a test done mostly fell in the high immunity group, indicating a likelihood of SARS-CoV-2 infection.

TABLE 13

| | | Samples April-June 2021 - No vaccine | | |
|---|---|---|---|---|
| | | Symptoms Positive (COVID19 | | |
| | | by symptoms) | | |
| | | PCR | PCR | PCR |
| | | Positive | Negative | N.D. |
| ≤63 | Undetectable | 3% | 31% | 17% |
| 64 to 93 | Cross reactivity | 7% | 29% | 17% |
| | level I | | | |
| 94 to 136 | Cross reactivity | 16% | 19% | 16% |
| | level II | | | |
| ≥137 | Pos-Covid | 74% | 21% | 51% |
| | average U IBBR | 191 | 100 | 148 |
| | SD U IBBR | 78 | 58 | 79 |
| | | N = 303 | N = 72 | N = 162 |

Table 14 shows measured IBBR values in subjects with no evidence of previous COVID-19 infection, stratified by their vaccination status. In non-vaccinated, asymptomatic patients, these subjects fell mostly with either groups 1 or 4, indicating either low immunity (no previous SARS-CoV-2 infection) or high immunity (likely experienced an asymptomatic SARS-CoV-2 infection). In vaccinated, asymptomatic patients most had high immunity to COVID-19, suggesting that vaccination does generally increase immunity.

TABLE 14

| | | Samples April-June 2021 | |
|---|---|---|---|
| | | Symptoms Neg, PCR Neg or PCR N.D. | |
| | | (no COVID19) | |
| | | | Vaccine (any Vaccine: |
| | | | one dose, two doses or |
| | | No Vaccine | single dose) |
| ≤63 | Undetectable | 31% | 7% |
| 64 to 93 | Cross reactivity | 18% | 11% |
| | level I | | |
| 94 to 136 | Cross reactivity | 17% | 23% |
| | level II | | |
| ≥137 | Pos-Covid | 34% | 58% |
| | average U IBBR | 115 | 160 |
| | SD U IBBR | 65 | 72 |
| | | N = 501 | N = 1132 |
| | | | p < 0.0001 |

Table 15 shows measured IBBR values in vaccinated subjects with no evidence of previous COVID-19 infection, stratified by the vaccination that they received. In each case, vaccinated subjects mostly fell within group 4, suggesting a high immunity to SARS-CoV-2 following vaccination. In most cases, the percentage of subjects in group 4 increased after the second dose and in all cases, the average IBBR value increase after the second dose.

TABLE 15

| | | Samples April-June 2021 | | | | | | |
|---|---|---|---|---|---|---|---|---|
| | | Symptoms Neg, PCR Neg, or PCR N.D. (no COVID19) | | | | | | |
| | | Coronavac | | Astrazenica | | | Pfizer | |
| | | One | Two | One | Two | J&J | One | Two |
| | | dose | doses | dose | doses | Single | dose | doses |
| ≤63 | Undetectable | 9% | 7% | 9% | 2% | 0% | 9% | 0% |
| 64 to 93 | Cross reactivity level I | 16% | 12% | 11% | 3% | 8% | 18% | 33% |
| 94 to 136 | Cross reactivity level II | 24% | 26% | 20% | 12% | 17% | 0% | 0% |
| ≥137 | Pos-Covid | 51% | 55% | 60% | 83% | 75% | 73% | 67% |
| | average U IBBR | 145 | 151 | 165 | 215 | 165 | 181 | 217 |
| | SD U IBBR | 68 | 63 | 84 | 78 | 47 | 76 | 92 |
| | | N = 144 | N = 589 | N = 161 | N = 99 | N = 12 | N = 11 | N = 3 |

Table 16 shows measured IBBR values in vaccinated subjects with no evidence of previous COVID-19 infection, stratified by the vaccination that they received. In each case, vaccinated subjects mostly fell within group 4, suggesting a high immunity to SARS-CoV-2 following vaccination. This data suggests that Astrazeneca provides higher immunity to SARS-CoV-2 than CoronaVac.

TABLE 16

| | | Samples April-June 2021 | |
|---|---|---|---|
| | | Symptoms Neg, PCR Neg, or PCR N.D. | |
| | | (no COVID19) | |
| | | CoronaVac | Astrazenica |
| | | (Two doses) | (Two doses) |
| ≤63 | Undetectable | 7% | 2% |
| 64 to 93 | Cross reactivity | 12% | 3% |
| | level I | | |
| 94 to 136 | Cross reactivity | 26% | 12% |
| | level II | | |
| ≥137 | Pos-Covid | 55% | 83% |
| | average U IBBR | 151 | 215 |
| | SD U IBBR | 63 | 78 |
| | | N = 589 | N = 99 |
| | | | p < 0.0001 |

Table 17 shows measured IBBR values in subjects with evidence of previous COVID-19 infection, stratified by their vaccination status. In non-vaccinated, symptomatic patients, these subjects fell mostly within group 4, indicating high immunity. In vaccinated, symptomatic patients most had high immunity to COVID-19. Indeed, the IBBR value and percentage of subjects in group 4 both increased after vaccination, suggesting that vaccination does generally increase immunity even in subjects with previous evidence of infection.

TABLE 17

| | | Samples April-June 2021 | |
|---|---|---|---|
| | | Symptoms Positive and PCR Positive | |
| | | (COVID19) | |
| | | | Vaccine (any vaccine: |
| | | | one doses, two doses |
| | | No Vaccine | or single) |
| ≤63 | Undetectable | 8% | 3% |
| 64 to 93 | Cross reactivity | 11% | 3% |
| | level I | | |
| 94 to 136 | Cross reactivity | 16% | 8% |
| | level II | | |
| ≥137 | Pos-Covid | 66% | 86% |
| | average U IBBR | 176 | 235 |
| | SD U IBBR | 81 | 88 |
| | | N = 465 | N = 198 |
| | | | p < 0.0001 |

Table 18 shows measured IBBR values in vaccinated subjects with evidence of previous COVID-19 infection, stratified by the vaccination that they received. In each case, vaccinated subjects mostly fell within group 4, suggesting a high immunity to SARS-CoV-2 following vaccination. In all cases, the percentage of subjects in group 4 increased after the second dose and in all cases, the average IBBR value increase after the second dose. This data suggests that Astrazeneca provides higher immunity to SARS-CoV-2 than CoronaVac.

TABLE 18

| | | Samples April-June 2021 | | | |
|---|---|---|---|---|---|
| | | Symptoms Positive and PCR Positive (COVID19) | | | |
| | | Coronavac | | Astrazenica | |
| | | One | Two | One | Two |
| | | dose * | doses * | dose ** | doses ** |
| ≤63 | Undetectable | 3% | 1% | 2% | 0% |
| 64 to 93 | Cross reactivity | 10% | 1% | 0% | 0% |
| | level I | | | | |
| 94 to 136 | Cross reactivity | 14% | 9% | 0% | 0% |
| | level II | | | | |
| ≥ 137 | Pos-Covid | 72% | 89% | 98% | 100% |
| | average U IBBR | 196 | 220 | 270 | 297 |
| | SD U IBBR | 73 | 75 | 87 | 66 |
| | | N = 29 | N = 79 | N = 47 | N = 14 |
| | | | * p = 0.137 | | ** p = 0.291 |

Table 19 shows measured IBBR values in vaccinated subjects with evidence of previous COVID-19 infection, stratified by the vaccination that they received. In each case, vaccinated subjects mostly fell within group 4, suggesting a high immunity to SARS-CoV-2 following vaccination. In all cases, the percentage of subjects in group 4 increased after the second dose and in all cases, the average IBBR value increase after the second dose.

TABLE 19

| | | Samples April-June 2021 | | | |
|---|---|---|---|---|---|
| | | Symptoms Positive and PCR Positive (COVID19) | | | |
| | | Coronavac | Astrazenica | Coronavac | Astrazenica |
| | | two doses | two doses * | one dose ** | one ** |
| ≤63 | Undetectable | 1% | 0% | 3% | 2% |
| 64 to 93 | Cross reactivity | 1% | 0% | 10% | 0% |
| | level I | | | | |
| 94 to 136 | Cross reactivity | 9% | 0% | 14% | 0% |
| | level II | | | | |
| ≥137 | Pos-Covid | 89% | 100% | 72% | 98% |
| | average U IBBR | 220 | 297 | 196 | 270 |
| | SD U IBBR | 75 | 66 | 73 | 87 |
| | | N = 79 | N = 14 | N = 29 | N = 47 |
| | | | * p = 0.0006 | | ** p = 0.0003 |

Table 20 shows measured IBBR values in vaccinated subjects stratified by their disease status. In each case, vaccinated subjects mostly fell within group 4, suggesting a high immunity to SARS-CoV-2 following vaccination. This data shows that immunity was higher in subjects that were both vaccinated and had received a previous positive COVID-19 test result.

TABLE 20

| | | Samples April-June 2021 | |
|---|---|---|---|
| | | Any Vaccine (one dose, two doses | |
| | | or single dose) | |
| | | | COVID19 |
| | | No COVID19 | (PCR positive) |
| ≤63 | Undetectable | 7% | 2% |
| 64 to 93 | Cross reactivity | 11% | 2% |
| | level I | | |
| 94 to 136 | Cross reactivity | 23% | 11% |
| | level II | | |
| ≥137 | Pos-Covid | 58% | 84% |
| | average U IBBR | 160 | 239 |
| | SD U IBBR | 72 | 90 |
| | | 1132 | 180 |
| | | | p < 0.0001 |

Table 21 shows exemplary conclusions that can be drawn following testing with the assay as described herein, where “A” and “B” describe example known indicators in a subject and potential test results. For example, in the first row, subject “A” is unvaccinated, has a previous positive COVID-19 PCR test, and a result of 190 IBBR units. It can be concluded that this subject has humoral immunity to SARS-CoV-2 and this is likely due to the confirmed COVID-19 infection confirmed with the PCR test result.

TABLE 21

| | | “A” | “B” | | |
|---|---|---|---|---|---|
| “A” | “B” | U - IBBR | U - IBBR | p value | Possible conclusions |
| COVID19 PCR | No COVID PCR | 190 | 100 | p < 0.0001 | PCR positive is an |
| Positive/No Vaccine | Negative/No Vaccine | | | | indicator of humoral |
| | | | | | immunity |
| COVID19 | No COVID | 166 | 121 | p < 0.0001 | Symptoms positive is an |
| Symptoms | Symptoms | | | | indicator of humoral |
| Positive/No Vaccine | Negative/No Vaccine | | | | immunity |
| No COVID19 + Any | No COVID19 | 160 | 115 | p < 0.0001 | Any vaccine is better |
| Vaccine (one doses, | | | | | then no Vaccine in no |
| two doses or single) | | | | | COVID19 patients |
| No COVID + | No COVID + | 151 | 145 | p = 0.3104 | One dose of Coronavac |
| Coronavac two doses | Coronavac one dose | | | | similar to two doses (No |
| | | | | | COVID19 patients |
| No COVID + | No COVID + | 215 | 165 | p < 0.0001 | Two doses of Astrazenica |
| Astrazenica two doses | Astrazenica one dose | | | | better than one dose (No |
| | | | | | COVID19 patients) |
| No COVID + | No COVID + | 215 | 151 | p < 0.0001 | Two doses of Astrazenica |
| Astrazenica two doses | Coronavac two doses | | | | better than two doses of |
| | | | | | Coronavac (No COVID19 |
| | | | | | patients) |
| COVID19 + Any | COVID19 + | 235 | 176 | p < 0.0001 | Any vaccine is better |
| Vaccine (one dose, | No Vaccine | | | | then no Vaccine in Pos- |
| two doses, single | | | | | COVID19 patients |
| dose) | | | | | |
| COVID19 + | COVID + | 220 | 196 | p = 0.137 | One dose of Coronavac is |
| Coronavac two doses | Coronavac one dose | | | | the same as two doses in |
| | | | | | Pos-COVID19 patients |
| COVID19 + | COVID19 + | 297 | 270 | p = 0.291 | One dose of Astrazenica |
| Astrazenica two doses | Astrazenica one dose | | | | is the same as two doses |
| | | | | | in Pos-COVID19 patients |
| COVID19 + | COVID19 + | 297 | 220 | p = 0.0006 | Two doses of Astrazenica |
| Astrazenica two doses | Coronavac two doses | | | | better than two doses of |
| | | | | | Coronavac in Pos- |
| | | | | | COVID19 patients |
| COVID19 + | COVID19 + | 270 | 196 | p = 0.0003 | One dose of Astrazenica |
| Astrazenica one dose | Coronavac one dose | | | | better than one dose of |
| | | | | | Coronavac in Pos- |
| | | | | | COVID19 patients |
| COVID19/No Vaccine | No COVID + | 191 | 151 | p < 0.0001 | COVID19 better then |
| | Coronavac two doses | | | | Coronavec (2 doses) in no |
| | | | | | COVID patients |
| No COVID19 + | COVID19/No Vaccine | 215 | 191 | p = 0.0078 | Two doses of Astrazenica |
| Astrazenica two doses | | | | | in no COVID19 better then |
| | | | | | pos COVID19 |
| No COVID19 + | No COVID19 + | 165 | 151 | p = 0.0267 | In no COVID patients, one |
| Astrazenica one dose | Coronavac two doses | | | | dose of Astazenica better |
| | | | | | then two doses of Coronavac |
| Any Vaccine pos | Any Vaccine after | 239 | 160 | p < 0.0001 | Vaccine after COVID19 |
| COVID19 | No COVID19 | | | | higher response then |
| | | | | | vaccine after no COVID19 |

The above disclosure generally describes the present invention. Although specific terms have been employed herein, such terms are intended in a descriptive sense and not for purposes of limitation.

Patent applications, patents, and publications are cited herein to assist in understanding the embodiments described. All such references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

Although specific embodiments of the invention have been described herein in detail, it will be understood by those skilled in the art that variations may be made thereto without departing from the spirit of the invention or the scope of the appended claims.

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

We claim:

1. An assay for determining immune status in a subject, the assay comprising:
a probe for measuring disease-specific antibodies in a sample from the subject to determine a unit score, wherein the unit score correlates to the immune status of the subject.

2. The assay of claim 1, wherein the unit score comprises an U-IBBR value.

3. The assay of claim 2, wherein the U-IBBR value is predictive of the immune status of the subject.

4. The assay of claim 2 or 3, wherein the U-IBBR value stratifies the subject into an immune status group.

5. The assay of claim 4, wherein the immune status group comprises a low immunity group and a high immunity group.

6. The assay of claim 4, wherein the immune status group comprises two or more of a low immunity group, a low-moderate immunity group, a moderate-high immunity group, and a high immunity group.

7. The assay of claim 4, wherein the immune status group comprises a low immunity group, a low-moderate immunity group, a moderate-high immunity group, and a high immunity group.

8. The assay of any one of claims 2 to 7, wherein the U-IBBR value ranges from 0 to about 300.

9. The assay of any one of claims 2 to 7, wherein the U-IBBR value ranges from 0 to about 200.

10. The assay of any one of claims 5 to 7, wherein the U-IBBR value ranges from 0 to about 90 in the low immunity group and the U-IBBR value ranges from about 110 to about 300 in the high immunity group.

11. The assay of any one of claims 5 to 7, wherein the U-IBBR value ranges from 20 to about 70 in the low immunity group and the U-IBBR value ranges from about 140 to about 300 in the high immunity group.

12. The assay of any one of claims 5 to 7, wherein the U-IBBR value ranges from about 30 to about 60 in the low immunity group and the U-IBBR value ranges from about 140 to about 300 in the high immunity group.

13. The assay of any one of claims 5 to 7, wherein the U-IBBR value ranges from 0 to about 50 in the low immunity group, the U-IBBR value ranges from about 51 to about 100 in the low-moderate immunity group, the U-IBBR value ranges from about 101 to about 140 in the moderate-high immunity group and the U-IBBR value ranges from about 141 to about 300 in the high immunity group.

14. The assay of any one of claims 5 to 7, wherein the U-IBBR value ranges from 0 to about 50 in the low immunity group, the U-IBBR value ranges from about 40 to about 100 in the low-moderate immunity group, the U-IBBR value ranges from about 70 to about 150 in the moderate-high immunity group and the U-IBBR value ranges from about 130 to about 300 in the high immunity group.

15. The assay of any one of claims 5 to 7, wherein the U-IBBR value ranges from 0 to about 90 in the low immunity group and the U-IBBR value ranges from about 110 to about 200 in the high immunity group.

16. The assay of any one of claims 5 to 7, wherein the U-IBBR value ranges from 20 to about 70 in the low immunity group and the U-IBBR value ranges from about 140 to about 200 in the high immunity group.

17. The assay of any one of claims 5 to 7, wherein the U-IBBR value ranges from about 30 to about 60 in the low immunity group and the U-IBBR value ranges from about 140 to about 200 in the high immunity group.

18. The assay of any one of claims 5 to 7, wherein the U-IBBR value ranges from 0 to about 50 in the low immunity group, the U-IBBR value ranges from about to about 100 in the low-moderate immunity group, the U-IBBR value ranges from about 101 to about 140 in the moderate-high immunity group and the U-IBBR value ranges from about 141 to about 200 in the high immunity group.

19. The assay of any one of claims 5 to 7, wherein the U-IBBR value ranges from 0 to about 50 in the low immunity group, the U-IBBR value ranges from about 40 to about 100 in the low-moderate immunity group, the U-IBBR value ranges from about 70 to about 150 in the moderate-high immunity group and the U-IBBR value ranges from about 130 to about 200 in the high immunity group.

20. The assay of any one of claims 1 to 19, wherein the probe comprises an antigen.

21. The assay of claim 20, wherein the antigen comprises a SARS-CoV-2 S1 or S2 protein or a variant or combination thereof.

22. The assay of claim 21, wherein the antigen comprises or consists of the polypeptide sequence of SEQ ID NO:1 or a variant thereof.

23. The assay of any one of claims 1 to 22, wherein the immune status is related to a disease of the subject.

24. The assay of claim 23, wherein the disease is an infectious disease or a non-infectious disease.

25. The assay of claim 24, wherein the infectious disease is selected from hepatitis, strep throat, urinary tract infections, tuberculosis, malaria, dengue fever, meningococcal disease, chloera, rabies, ebola, influenza, coronavirus disease-2019 (COVID-19), herpes, respiratory infection, Severe Acute Respiratory Syndrome (SARS), Middle East Respiratory Syndrome (MERS), mumps, measles, rubella, polio, small pox, human immunodeficiency virus (HIV), acquired immune deficiency syndrome (AIDS), pneumonia, Lyme disease, anthrax, tetanus, cholera, plague, diptheria, chlamydia or malaria.

26. The assay of claim 24, wherein the infectious disease is coronavirus disease-2019 (COVID-19).

27. The assay of any one of claims 1 to 26, wherein the sample is selected from blood, blood plasma, blood serum, hemolysate, spinal fluid, urine, lymph, synovial fluid, saliva, sperm, amniotic fluid, lacrimal fluid, cyst fluid, sweat gland secretion, or bile.

28. The assay of any one of claims 1 to 26, wherein the sample is blood.

29. A method for determining immune status in a subject, the method comprising:
measuring disease-specific antibodies in a sample; and
determining a unit score based on the disease-specific antibodies measured; wherein the unit score correlates to the immune status of the subject.

30. The method of claim 29, wherein the unit score comprises an U-IBBR value.

31. The method of claim 30, wherein the U-IBBR value is predictive of the immune status of the subject.

32. The method of any one of claims 29 to 31, wherein the U-IBBR value stratifies the subject into an immune status group.

33. The method of claim 32, wherein the immune status group comprises a low immunity group and a high immunity group.

34. The method of claim 32, wherein the immune status group comprises two or more of a low immunity group, a low-moderate immunity group, a moderate-high immunity group, and a high immunity group.

35. The method of claim 32, wherein the immune status group comprises a low immunity group, a low-moderate immunity group, a moderate-high immunity group, and a high immunity group.

36. The method of any one of claims 30 to 35, wherein the U-IBBR value ranges from 0 to about 300.

37. The method of any one of claims 30 to 35, wherein the U-IBBR value ranges from 0 to about 200.

38. The method of any one of claims 33 to 35, wherein the U-IBBR value ranges from 0 to about 90 in the low immunity group and the U-IBBR value ranges from about 110 to about 300 in the high immunity group.

39. The method of any one of claims 33 to 35, wherein the U-IBBR value ranges from 20 to about 70 in the low immunity group and the U-IBBR value ranges from about 140 to about 300 in the high immunity group.

40. The method of any one of claims 33 to 35, wherein the U-IBBR value ranges from about 30 to about 60 in the low immunity group and the U-IBBR value ranges from about 140 to about 300 in the high immunity group.

41. The method of any one of claims 33 to 35, wherein the U-IBBR value ranges from 0 to about 50 in the low immunity group, the U-IBBR value ranges from about 51 to about 100 in the low-moderate immunity group, the U-IBBR value ranges from about 101 to about 140 in the moderate-high immunity group and the U-IBBR value ranges from about 141 to about 300 in the high immunity group.

42. The method of any one of claims 33 to 35, wherein the U-IBBR value ranges from 0 to about 50 in the low immunity group, the U-IBBR value ranges from about 40 to about 100 in the low-moderate immunity group, the U-IBBR value ranges from about 70 to about 150 in the moderate-high immunity group and the U-IBBR value ranges from about 130 to about 300 in the high immunity group.

43. The method of any one of claims 33 to 35, wherein the U-IBBR value ranges from 0 to about 90 in the low immunity group and the U-IBBR value ranges from about 110 to about 200 in the high immunity group.

44. The method of any one of claims 33 to 35, wherein the U-IBBR value ranges from 20 to about 70 in the low immunity group and the U-IBBR value ranges from about 140 to about 200 in the high immunity group.

45. The method of any one of claims 33 to 35, wherein the U-IBBR value ranges from about 30 to about 60 in the low immunity group and the U-IBBR value ranges from about 140 to about 200 in the high immunity group.

46. The method of any one of claims 33 to 35, wherein the U-IBBR value ranges from 0 to about 50 in the low immunity group, the U-IBBR value ranges from about 51 to about 100 in the low-moderate immunity group, the U-IBBR value ranges from about 101 to about 140 in the moderate-high immunity group and the U-IBBR value ranges from about 141 to about 200 in the high immunity group.

47. The method of any one of claims 33 to 35, wherein the U-IBBR value ranges from 0 to about 50 in the low immunity group, the U-IBBR value ranges from about 40 to about 100 in the low-moderate immunity group, the U-IBBR value ranges from about 70 to about 150 in the moderate-high immunity group and the U-IBBR value ranges from about 130 to about 200 in the high immunity group.

48. The method of any one of claims 29 to 47, wherein the disease-specific antibodies are measured using a probe.

49. The method of claim 48, wherein the probe comprises an antigen.

50. The method of claim 49, wherein the antigen comprises a SARS-CoV-2 S1 or S2 protein or a variant or combination thereof.

51. The method of claim 50, wherein the antigen comprises or consists of the polypeptide sequence of SEQ ID NO:1 or a variant thereof.

52. The method of any one of claims 29 to 51, wherein the immune status is related to a disease of the subject.

53. The method of claim 52, wherein the disease is an infectious disease or a non-infectious disease.

54. The method of claim 53, wherein the infectious disease is selected from hepatitis, strep throat, urinary tract infections, tuberculosis, malaria, dengue fever, meningococcal disease, chloera, rabies, ebola, influenza, coronavirus disease-2019 (COVID-19), herpes, respiratory infection, Severe Acute Respiratory Syndrome (SARS), Middle East Respiratory Syndrome (MERS), mumps, measles, rubella, polio, small pox, human immunodeficiency virus (HIV), acquired immune deficiency syndrome (AIDS), pneumonia, Lyme disease, anthrax, tetanus, cholera, plague, diptheria, chlamydia or malaria.

55. The method of claim 53, wherein the infectious disease is coronavirus disease-2019 (COVID-19).

56. The method of any one of claims 29 to 55, wherein the sample is selected from blood, blood plasma, blood serum, hemolysate, spinal fluid, urine, lymph, synovial fluid, saliva, sperm, amniotic fluid, lacrimal fluid, cyst fluid, sweat gland secretion, or bile.

57. The method of any one of claims 29 to 55, wherein the sample is blood.

58. The method of any one of claims 29 to 57, wherein the method is useful for predicting the likelihood of being infected with a pathogen.

59. The method of any one of claims 29 to 57, wherein the method is useful for determining vaccination status for the disease.

60. The method of any one of claims 29 to 57, wherein the method is useful for determining whether the subject should be administered an RNA vaccine against the disease.

61. The method of any one of claims 29 to 57, further comprising vaccinating the subject if the immune status has a predetermined unit score value.

62. The method of claim 61, wherein the predetermined unit score value ranges from 0 to about 140.

63. A method for predicting the likelihood of being infected with a pathogen, the method comprising:
measuring pathogen-specific antibodies in a sample; and
determining a unit score based on the pathogen-specific antibodies measured; wherein the unit score predicts the likelihood of being infected with the pathogen.

64. A method for determining vaccination status for a disease, the method comprising:
measuring disease-specific antibodies in a sample from a subject; and
determining a unit score based on the disease-specific antibodies measured; wherein the unit score correlates to vaccination status.

65. A method for determining whether a subject should be administered an RNA vaccine against a disease, the method comprising:
measuring disease-specific antibodies in a sample from a subject; and
determining a unit score based on the disease-specific antibodies measured; wherein the unit score predicts whether the subject should receive the RNA vaccine.

66. A method for vaccinating a subject against a disease, the method comprising:
measuring disease-specific antibodies in a sample from the subject;
determining a unit score based on the disease-specific antibodies measured; wherein the unit score correlates to an immune status of the subject; and
vaccinating the subject if the immune status has a predetermined unit score value.

67. A method for measuring vaccine efficacy, the method comprising:
measuring disease-specific antibodies in a sample from a vaccinated subject; and
determining a unit score based on the disease-specific antibodies measured; wherein the unit score predicts the efficacy of the vaccine.

68. The method of any one of claims 29 to 67, further comprising a step of obtaining the sample from the subject.

69. An antigen for detecting and/or generating SARS-CoV-2 antibodies, wherein the antigen comprises the SARS-CoV-2 S1 and S2 proteins or variants thereof, wherein the S1 and S2 proteins or variants adopt a substantially native conformation.

70. An antigen for detecting and/or generating SARS-CoV-2 antibodies, wherein the antigen comprises a spike protein conformational epitope or variant thereof, wherein the epitope is not present in S1 or S2 alone.

71. An antigen comprising or consisting of the polypeptide sequence of SEQ ID NO:1:

| | |
|---|---|
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfcndpf lgvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqdvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkfngltv lpplltdemi aqytsallag titsgwtfga gaalqipfam |
| 901 | qmayrfngig vtqnvlyenq klianqfnsa igkiqdslss tasalgklqd vvnqnaqaln |
| 961 | tlvkqlssnf gaissvlndi lsrldkveae vqidrlitgr lqslqtyvtq qliraaeira |
| 1021 | sanlaatkms ecvlgqskrv dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyeq |

or a variant thereof.

72. The antigen of claim 71, further comprising one or more stabilizing residues, such as stabilizing proline residues, for example at the C-terminus.

73. The antigen of any one of claims 69 to 72, wherein the variant comprises at least about 50%, 60%, 70%, 80%, 90%, 95%, or 99% sequence identity to the native S1 and S2 proteins, the native conformational epitope, or SEQ ID NO:1.

74. The antigen of any one of claims 69 to 73, further comprising the SARS-CoV-2 trunk protein.

75. The antigen of any one of claims 69 to 74, wherein the antigen is N-glycosylated.

76. The antigen of claim 75, wherein the antigen is N-glycosylated at 50%, 60%, 70%, 80%, 90%, 95%, or 99%, or more, or 100% of the native N-glycosylation sites.

77. The antigen of any one of claims 69 to 76, wherein the antigen is trimeric.

78. A diagnostic assay comprising the antigen of any one of claims 69 to 77.

79. The assay of claim 78, wherein the assay is an ELISA, a lateral flow assay, or a chemiluminescence immunoassay.

80. The assay of claim 78 or 79, wherein the assay detects IgG, IgM, and/or IgA.

81. The assay of any one of claims 78 to 80, wherein the assay detects antibodies in a sample selected from blood, blood plasma, blood serum, hemolysate, spinal fluid, urine, lymph, synovial fluid, saliva, sperm, amniotic fluid, lacrimal fluid, cyst fluid, sweat gland secretion, and bile.

82. A vaccine against SARS-CoV-2 comprising the antigen of any one of claims 69 to 81.

83. The vaccine of claim 82, further comprising an adjuvant.
